(19) United States
(12) Patent Application Publication
(10) Pub. No.: US 2020/0405195 A1
(43) Pub. Date: Dec. 31, 2020

(54) COMPUTATIONAL FABRICS FOR MONITORING HUMAN JOINT MOTION

(71) Applicant: Trustees of Dartmouth College, Hanover, NH (US)

(72) Inventors: Ruibo Liu, West Lebanon, NH (US); Qijia Shao, West Lebanon, NH (US); Siqi Wang, Shanghai (CN); Christina Ru, Bellaire, TX (US); Devin Balkcom, Hanover, NH (US); Xia Zhou, Hanover, NH (US)

(73) Assignee: Trustees of Dartmouth College, Hanover, NH (US)

(21) Appl. No.: 16/911,877

(22) Filed: Jun. 25, 2020

Related U.S. Application Data
(60) Provisional application No. 62/866,184, filed on Jun. 25, 2019.

(51) Int. Cl. A61B 5/11; A61B 5/00; D02G 3/02; D02G 3/32; D02G 3/44

(52) U.S. Cl. CPC A61B 5/1121; A61B 5/7275; A61B 5/7246; D02G 3/441; D02G 3/02; D02G 3/32; D02G 3/448; A61B 5/6813

(57) ABSTRACT

In an embodiment, the present disclosure pertains to a method of determining an angular motion in a subject. The method generally includes one or more of the following steps of: (1) applying a wearable system to a body region of the subject; (2) utilizing the wearable system to sense one or more parameters; and (3) correlating the one or more parameters to the angular motion in the subject. In an additional embodiment, the present disclosure pertains to a wearable system for determining angular motion in a subject. Generally, the wearable sensor includes one or more fabrics for sensing one or more parameters of a body region of a subject.

### CROSS-REFERENCE TO RELATED APPLICATIONS

This application claims priority to U.S. Provisional Patent Application No. 62/866,184, filed on Jun. 25, 2019. The entirety of the aforementioned application is incorporated herein by reference.

### STATEMENT REGARDING FEDERALLY SPONSORED RESEARCH

This invention was made with government support under U.S. Pat. No. 1,822,819 awarded by the National Science Foundation. The government has certain rights in the invention.

### BACKGROUND

Prior efforts to utilize electronic textiles for monitoring angular motion have had numerous limitations. Such limitations have included coarse sensing capabilities, requirements for specialized fabrics, and requirements to embed electronic devices into textiles. Various embodiments of the present disclosure address the aforementioned limitations.

### SUMMARY

In an embodiment, the present disclosure pertains to a method of determining an angular motion in a subject. The method generally includes one or more of the following steps of: (1) applying a wearable system to a body region of the subject; (2) utilizing the wearable system to sense one or more parameters; and (3) correlating the one or more parameters to the angular motion in the subject.

In an additional embodiment, the present disclosure pertains to a wearable system for determining angular motion in a subject. In general, the wearable sensor includes one or more fabrics for sensing one or more parameters of a body region of a subject.

### DESCRIPTION OF THE DRAWINGS

FIG. 1A depicts a method of determining an angular motion according to an aspect of the present disclosure.

FIG. 1B depicts a wearable system to determine an angular motion according to an aspect of the present disclosure.

FIG. 2 illustrates a test platform configuration according to an aspect of the present disclosure.

FIGS. 3A and 3B illustrate the change of fabric resistance as the fabric is stretched. Resistance sharply increases due to the decrease in yarn contact points during fiber slipping, and then gradually decreases under increasing contact pressure during yarn stretching. FIG. 3A shows the overall trend. FIG. 3B shows the various phases during a stretch.

FIG. 4A illustrates resistance change of stretchable conductive fabric when it is stretched along its wale or course direction.

FIGS. 4B and 4C are microscope images on fabric's micro-structure under 30% strain along the course and wale direction, respectively.

FIGS. 5A and 5B illustrate the impact of fabric size configuration on its sensitivity to stretch. FIG. 5A shows varying width. FIG. 5B shows varying length.

FIG. 6 illustrates elastic hysteresis. As shown in FIG. 6, resistance change follows a different curve during loading/extending and unloading/retracting.

FIGS. 7A and 7B illustrate impact of stress relaxation (FIG. 7A) and the instability of resistance change in 160 rounds of stretching (FIG. 7B).

FIG. 8 illustrates a system design overview according to an aspect of the present disclosure.

FIG. 9 illustrates examination of the resistance change of various pressure fabrics under pressure.

FIG. 10 illustrates detection of motion states.

FIG. 11 illustrates a Quantile-Quantile (Q-Q) plot of sample data vs. the standard Gaussian distribution.

FIG. 12 illustrates approximating a strain fabric using a spring-dashpot model according to an aspect of the present disclosure.

FIG. 13 illustrates the anatomy of an elbow joint.

FIGS. 14A, 14B and 14C illustrate inferring the rotational angle based on the stretch lengths sensed by two strain fabrics. FIG. 14A shows pressure fabric on the first layer while two strain fabrics are on the second layer. FIG. 14B denotes the orthogonal distances between strain sensors and virtual rotation axis as R1 and R2. The two radiuses, the virtual axis and skin surface slope will form a trapezoid cross-section. The flat surface is also plotted for reference. FIG. 14C shows the rotation of the cross-section will form a circular truncated cone whose top surface radius is the deformation of strain fabric 2 and base radius is that of strain fabric 1. Its height can be computed with distance d and angle cp. FIG. 14A shows two layers of sensing fabrics on the elbow: pressure sensor and strain sensors. FIG. 14B shows a trapezoid cross-section (grey) formed around the virtual rotation axis. FIG. 14C shows a circular truncated cone formed by the rotation of cross-section.

FIGS. 15A, 15B, and 15C illustrate integrating strain and pressure sensing fabrics into regular elastic fabrics according to aspects of the present disclosure. FIG. 15A shows Layer 1 (inner layer). FIG. 15B shows Layer 2 (external layer). FIG. 15C shows the final look.

FIGS. 16A, 16B, and 16C illustrate that one-layer strain fabric leads to mechanical instability over repeated runs (FIG. 16A), while a two-layer structure (FIG. 16B) provides a much more stable output (FIG. 16C). FIG. 16A shows a one-layer structure. FIG. 16B shows fabricating two-layer strain fabric. FIG. 16C shows a two-layer structure.

FIGS. 17A, 17B, 17C, and 17D illustrate strain and pressure fabrics. FIG. 17A shows a two-layer strain fabric. FIG. 17B shows a two-layer strain fabric (side). FIG. 17C shows a two-layer pressure fabric. FIG. 17D shows pressure fabric components.

FIGS. 18A, 18B, and 18C illustrate overall accuracy in inferring joint angles, including the average accuracy of each participant (FIG. 18A), the impact of arm size on angular errors (FIG. 18B), and the overall accuracy for each gender (FIG. 18C).

FIGS. 19A, 19B, and 19C illustrate accuracy of inferring joint angles under controlled motions, where the pause between adjacent movement (FIG. 19A), movement speed (FIG. 19B), and flexion angles (FIG. 19C) is varied.

FIG. 20 illustrates accuracy of inferring joint angles under three free-form motions.

FIG. 21 illustrates impact of fabric displacement on sensing accuracy.

FIG. 22 illustrates impact of wash on fabric's sensing performance.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Human body joints are used for actuating body motion. Accurate and continuous monitoring of their rotational movement is useful for physical rehabilitation, motion training, motion coaching, sports analytics, and human-robot or human-computer interactions. For rehabilitation patients with joint injuries or chronic joint pains, day-to-day measurement of joint's angular motion helps doctors assess the effectiveness of medical and physical treatments, since joint and muscle repair takes long enough that it is impractical for expert therapists to remain with patients at all times For students learning precise motions from instructors in an educational context (e.g., athletic coaching, yoga training, or learning a complex surgical procedure), monitoring joint angles allows instructors to analyze detailed joint movements and provide fine-grained corrections and feedback.

These applications, to name only a few, need joint motion sensing systems that are portable, comfortable for long-time wear, and capable of sensing subtle motion. Existing technologies for motion capture, however, still fall short in meeting these requirements.

Currently, portable systems either achieve coarse sensing granularity by classifying a limited set of gestures or poses. Thus, current systems cannot track arbitrary motion. Moreover, current systems require users to constantly wear rigid electrical sensors right around the joint area, where such placement inflexibility often causes the system to be burdensome to wear.

In sum, a need exists for more effective systems and methods for determining an angular motion in a subject. Various embodiments of the present disclosure address the aforementioned need.

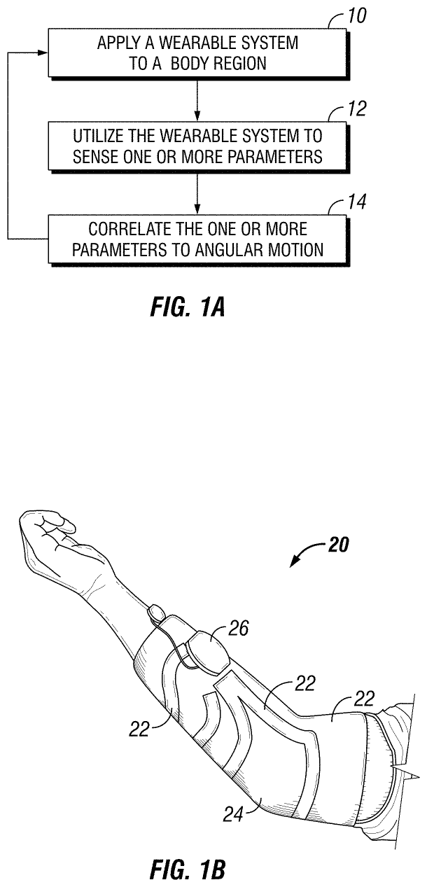

In some embodiments, the present disclosure pertains to methods of determining an angular motion in a subject. In some embodiments illustrated in FIG. 1A, the methods of the present disclosure generally include one or more of the following steps of: applying a wearable system to a body region of the subject (step 10), utilizing the wearable system to sense one or more parameters (step 12), and correlating the one or more parameters to the angular motion in the subject (step 14). In some embodiments, the methods of the present disclosure are repeated until a desired number of data points for the angular motion are obtained. In some embodiments, the methods of the present disclosure can be conducted in real time. Furthermore, in some embodiments, the methods of the present disclosure are utilized for rehabilitation.

Additional embodiments of the present disclosure pertain to wearable systems for determining angular motion in a subject. The wearable systems of the present disclosure generally include one or more fabrics for sensing one or more parameters. In some embodiments illustrated in FIG. 1B, a wearable system 20 of the present disclosure include one or more fabrics 22, which wrap around a joint 24, and are operable to sense the one or more parameters associated with movement of the joint 24. In some embodiments, as illustrated in FIG. 1B, the wearable system additionally includes a micro-controller 26.

As set forth in more detail herein, the methods and wearable systems of the present disclosure can have numerous embodiments. For instance, the methods of the present disclosure can sense various parameters and determine various angular motions in different body regions utilizing numerous correlation methods. Additionally, the wearable systems of the present disclosure can include various types of fabrics, have various structures, and can be applied to body regions in various ways.

Applying a Wearable System to a Body Region of a Subject

The methods of the present disclosure can apply numerous wearable systems to various body regions of various subjects. For instance, in some embodiments, the subject is a human being. In some embodiments, the subject is a human being suffering from a joint-related disorder or condition.

In some embodiments, the body region to which the wearable system is applied can include, without limitation, joints, muscles, a body extremity, a prehensile, an appendage of a body, an upper limb, a lower limb, a pivot joint, a hinge joint, a saddle joint, a ball-and-socket joint, a planar joint, an ellipsoidal joint, an elbow joint, a knee joint, an ankle joint, a wrist joint, a neck joint, a finger joint, a foot joint, a toe joint, a leg joint, an arm joint, and combinations thereof.

In some embodiments, the wearable system is applied to a body region by wrapping the wearable system around the body region. In some embodiments, the wearable system is applied to a body region by wrapping the wearable system around a joint.

Sensing One or More Parameters

The methods of the present disclosure can utilize the wearable systems disclosed herein to sense one or more parameters. For instance, in some embodiments, the one or more parameters include, without limitation, resistance, pressure, deformation, skin deformation, strain, stress relaxation, and combinations thereof. In some embodiments, the one or more parameters include pressure and deformation.

In some embodiments, the sensing includes measuring strain of the wearable system. In some embodiments, the measuring includes sensing a change in resistance in the wearable system. In some embodiments, the change in resistance is caused by an alteration of a contact point in a portion of yarn of the wearable system. In some embodiments, the change in resistance is caused by an alteration of contact pressure in a portion of yarn of the wearable system. In some embodiments, the change in resistance is caused by an alteration of contact points and contact pressure of a portion of yarn of the wearable system.

In some embodiments, the sensing includes measuring a change in pressure in the wearable system. In some embodiments, the change in pressure is caused by at least one of yarn stretching, movement of the subject, movement at the body region, movement at a joint at the body region, and combinations thereof.

Correlation of Parameters to Angular Motion

Various methods may be utilized to correlate one or more sensed parameters to angular motion. For instance, in some embodiments, the correlating includes at least one of predicting angular motion, measuring angular motion, reconstructing angular motion, modeling angular motion, and combinations thereof.

In some embodiments, the correlating includes using a model. In some embodiments, the model includes, without limitation, a model to compensate for stress relaxation during a motionless state of the subject, a model to characterize a geometric relationship between skin deformation and joint angle, a material science model, a biomechanical model, and combinations thereof.

In some embodiments, the correlating includes one or more of the following steps of: (1) monitoring resistance of the wearable system to infer muscle strain caused by joint motion; (2) recovering joint angle based, at least in part, on the monitoring; and (3) compensating for stress relaxation during a motionless state. In some embodiments, the correlating further includes one or more of the following steps of: (1) characterizing a geometric relationship between skin deformation and joint angle; and (2) inferring joint rotational angle based, at least in part, on the characterization. In some embodiments, the correlating further includes one or more of the following steps of: (1) characterizing a geometric relationship between skin deformation, fabric strain, and pressure properties and joint angle; and (2) inferring joint rotational angle based, at least in part, on the characterization.

The correlation of the one or more parameters can be utilized to determine various types of angular motion. For instance, in some embodiments, the angular motion includes, without limitation, rotational motion, arbitrary motion, coarse motion, flexion, extension, motionless states, joint motion, joint rotation, muscle movement, rotational angle, joint rotational angle, and combinations thereof. In some embodiments, the angular motion is at least one of flexion and extension.

Fabrics and Components of the Wearable Systems

As set forth in more detail herein, the methods of the present disclosure can utilize various wearable systems having various fabrics and components. For instance, in some embodiments, the wearable system includes a fabric. In some embodiments, the fabric includes, without limitation, a stretchable fabric, a conductive fabric, a stretchable and conductive fabric, a pressure sensitive fabric, a knitted fabric, and combinations thereof.

In some embodiments, the fabric is a knitted fabric constructed by a continuous loop of yarns. In some embodiments, the fabric is a knitted fabric constructed by stretchable threads. In some embodiments, the fabric includes yarns coated with a conductive material to increase conductivity. In some embodiments, the fabric includes a mixture of nylon and elastic fiber. In some embodiments, the fabric is capable of being stretched by up to 100% along a length/course direction and up to 60% along a width/wale direction.

In some embodiments, the fabric has a structure. In some embodiments, the structure can include, without limitation, a soft and natural sensing layer that is operable to be wrapped around the body region of the subject to reconstruct angles of the body region at a fine granularity. In some embodiments, the fabric includes a thin layer operable to dissipate heat. In some embodiments, the fabric lacks a rigid electrical sensor. In some embodiments, the rigid electrical sensor is an electrode. In some embodiments, the fabric lacks the electrode. In some embodiments, the fabric is operable to sense at least one parameter of the one or more parameters.

In some embodiments, the fabric includes a plurality of fabrics. In some embodiments, each fabric of the plurality of fabrics is operable to sense at least one parameter of the one or more parameters. In some embodiments, the plurality of fabrics are operable to sense skin deformation and pressure caused by joint movement.

In some embodiments, the plurality of fabrics include a first fabric operable to sense a first parameter of the one or more parameters and a second fabric operable to sense a second parameter of the one or more parameters. In some embodiments, the first fabric is operable to sense deformation and the second fabric is operable to sense pressure. In some embodiments, the first fabric reacts to different levels of strain with varying resistance to sense deformation and the second fabric augments the first fabric by sensing pressure from the body region during motion. In some embodiments, the motion is joint movement.

In some embodiments, the fabric includes one or more layers. In some embodiments, the one or more layers include a first layer that faces a portion of skin and a second layer positioned above the first layer. In some embodiments, the first layer senses pressure and the second layer senses deformation. In some embodiments, the one or more layers include an insulative layer. In some embodiments, the insulative layer protects at least a portion of the fabric. In some embodiments, the insulative layer protects at least a portion of the fabric from bodily fluids, for example, but not limited to, perspiration.

In some embodiments, the fabric includes a conductive and stretchable fabric and a pressure sensitive fabric. In some embodiments, the conductive and stretchable fabric senses deformation. In some embodiments, the pressure sensitive fabric senses pressure. In some embodiments, the sensed deformation and sensed pressure are correlated to joint rotational angle.

Additionally, as set forth in more detail herein, the wearable systems of the present disclosure can include various components. For instance, in some embodiments the wearable systems can further include a microcontroller. In some embodiments, the microcontroller obtains data relating to the one or more parameters. In some embodiments, the microcontroller resides outside of a fabric of the wearable system. In some embodiments, the microcontroller is embedded in a button.

Wearable Systems

Additional embodiments of the present disclosure pertain to wearable systems described herein. The wearable systems of the present disclosure generally include one or more fabrics for sensing or more parameters of a body region in a subject to determine angular motion.

The wearable systems of the present disclosure can include, without limitation, various components of the wearable systems as described in detail herein. For instance, in some embodiments, the one or more fabrics can include, without limitation, one or more fabrics as described in detail herein. In some embodiments, the one or more parameters operable to be sensed by the one or more fabrics can include, without limitation, one or more parameters as described in detail herein. In some embodiments, the angular motion can include, without limitation, one or more angular motions as described in detail herein. In some embodiments, the wearable systems of the present disclosure can further include a microcontroller as described in detail herein.

Applications and Advantages

Prior efforts to utilize electronic textiles (e-textiles) for monitoring angular motion have numerous limitations. Such limitations include coarse sensing capabilities, the requirement for specialized fabrics, or the requirement to embed electronic devices into textiles. In some embodiments, the wearable systems of the present disclosure differ from prior e-textiles in that they rely on off-the-shelf, low-cost fabrics, and achieve qualitative advances in sensing granularity.

Moreover, in some embodiments, the methods and wearable systems of the present disclosure enable everyday fabrics to be an unobtrusive sensing media to monitor angular motion (e.g., joint motion) continuously and/or in real time. In some embodiments, the methods and wearable systems of the present disclosure require neither infrastructure support nor rigid electrical sensors on body regions, such as joints. As such, the methods and wearable systems of the present disclosure are portable, comfortable for long-term wear, low-cost, and low-power.

In some embodiments, the methods and wearable systems of the present disclosure address challenges originating from intrinsic properties of elastic materials and practical uses on the body. These challenges include elastic properties, such as, but not limited to, hysteresis and stress relaxation, as well as user diversity and motion artifact.

In some embodiments, the combined use of stretchable and pressure fabrics deal with the aforementioned challenges to disambiguate motion states. For instance, in some embodiments, models from material science are applied to compensate for stress relaxation during motionless states. Additionally, in some embodiments, models from bio-mechanics are leveraged to characterize the geometric relationship between skin deformation and joint angle. In some embodiments, the design of two straps of strain fabrics as dual sensors help cancel out the impact of user diversity and fabric offset caused by motion.

As such, the methods and wearable systems of the present disclosure can have numerous applications. For instance, in some embodiments, the wearable systems and methods of the present disclosure can be utilized in physical rehabilitation. Moreover, in some embodiments, the wearable systems and methods of the present disclosure can be utilized in motion training.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Reconstructing Human Joint Motion with Computational Fabrics

This Example describes reconstruction of human joint motion with computational fabrics.

Accurate and continuous monitoring of joint rotational motion is used for a wide range of applications such as physical rehabilitation and motion training. Existing motion capture systems, however, either need instrumentation of the environment, or fail to track arbitrary joint motion, or impose wearing discomfort by requiring rigid electrical sensors right around the joint area. This Example studies the use of everyday fabrics as a flexible and soft sensing medium to monitor joint angular motion accurately and reliably.

Specifically, Applicants focus on the primary use of conductive stretchable fabrics to sense the skin deformation during joint motion and infer the joint rotational angle. Applicants tackle challenges of fabric sensing originated by the properties of elastic materials by leveraging two types of sensing fabric and characterizing their properties based on models in material science. Applicants apply models from bio-mechanics to infer joint angles and propose the use of dual strain sensing to enhance sensing robustness against user diversity and fabric position offsets. Applicants fabricate prototypes using off-the-shelf fabrics and micro-controller. Experiments with ten participants show 9.690 median angular error in tracking joint angle and its sensing robustness across various users and activities.

### Example 1.1. Introduction

Human body joints are used for actuating body motion. Accurate and continuous monitoring of their rotational movement is useful for physical rehabilitation, motion training/coaching, sports analytics, and human-robot or human-computer interactions. For rehabilitation patients with joint injuries or chronic joint pains, day-to-day measurement of joint's angular motion helps doctors assess the effectiveness of medical and physical treatments, since joint and muscle repair takes long enough that it is impractical for expert therapists to remain with patients at all times; for students learning precise motions from instructors in the educational context (e.g., athletic coaching, yoga training, learning a complex surgical procedure), monitoring joint angles allows instructors to analyze detailed joint movements and provide fine-grained corrections and feedback.

All these applications need joint motion sensing systems that are portable, comfortable for long-time wear, and capable of sensing subtle motion. Existing technologies for motion capture, however, still fall short in meeting these requirements. High-end systems such as VICON or Kinect require heavy instrumentation of the environment (e.g., setting up multiple infrared cameras). More portable systems either achieve coarse sensing granularity by classifying a limited set of gestures/poses and thus cannot track arbitrary motion, or require users to constantly wear rigid electrical sensors right around the joint area, where such placement inflexibility often causes the system burdensome to wear.

In this Example, Applicants consider the use of everyday fabrics as an unobtrusive sensing medium to continuously and accurately monitor joint angular motion. Requiring neither infrastructure support nor rigid electrical sensors on the joint, Applicants' approach relies on fabrics/textile alone, as a soft and natural sensing layer around joints, to reconstruct the angles of body joints at a fine granularity. While the concept of electronic textile (e-textile) has been proposed in prior studies for various applications, prior works either offer coarse sensing capabilities or still require embedding electronics into textiles by using textile as a substrate for attachment of sensors.

Applicants aim to advance the state-of-the-art by achieving qualitative advances in sensing capability and reliability, and more importantly, by focusing on the use of off-the-shelf, low-cost (e.g., $50) fabrics alone for motion sensing without extra electrical sensors. Such a minimalist sensing approach renders the sensing system comfortable to wear, low-power, and low-cost.

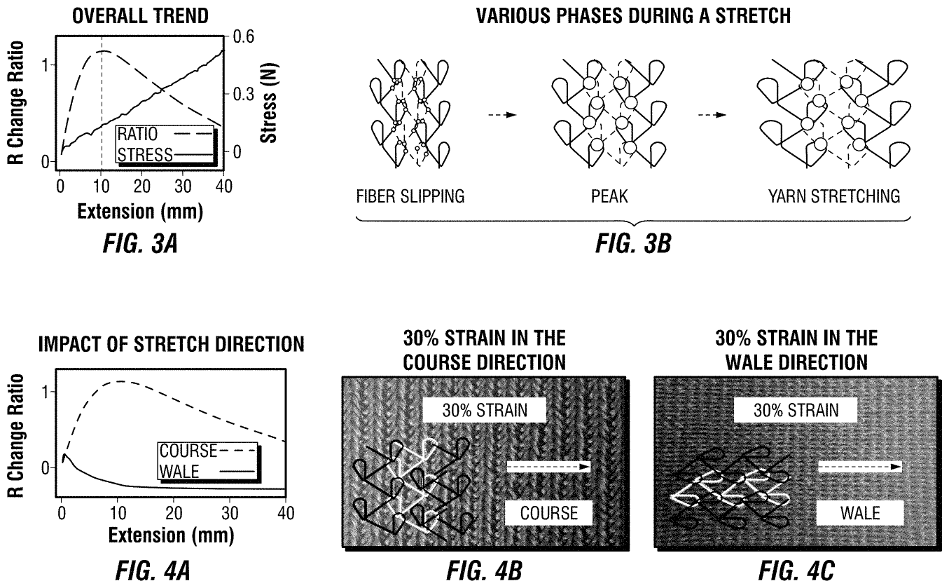

Specifically, Applicants study primarily the use of conductive stretchable fabrics with knitted structures. As shown in FIG. 3B, a knitted fabric is constructed by a continuous loop of yarns. Made of conductive stretchable threads, the knitted fabric reacts to different levels of strain with varying resistance. The change in resistance is caused by alterations of the yarns' contact points and contact pressure under tension. This property can be exploited to sense body joint motion, where Applicants wrap a joint with conductive stretchable fabrics (also serving as a joint protective wrap commonly worn during exercises for joint protection). Joint rotation and muscle movement cause skin deformation and thus strain of the fabric. By continuously monitoring the fabric's resistance, Applicants can infer the muscle strain caused by the current joint motion, and thus recover the joint angle. The system only requires a micro-controller fetching data on fabric resistance through conductive threads. The micro-controller can be placed away from the joint for greater comfort flexibility. As an example, it can be embedded into a button to hide its appearance and it can be easily detached when necessary.

To realize this conceptually simple idea as a practical system providing accurate and robust motion sensing, Applicants are confronted with several challenges. First, with off-the-shelf stretchable fabrics, the resistance change and the level of strain do not exhibit a one-to-one mapping, and even worse, their relationship depends on the type of motion (e.g., flexion/loading or extension/unloading), caused by the hysteresis intrinsic to elastic materials. It results in ambiguities in inferring skin deformation solely based on the observed changes in fabric resistance. Second, fabric as a strain sensor does not provide a stable output even under a constant strain. This is a phenomenon common in stretchable materials and referred to as the stress relaxation in material science. Such relaxation further increases the ambiguity in inferring skin deformation and leads to errors that accumulate over time. Third, even with perfect derivation of skin deformation, inferring the actual joint angle is still nontrivial. For one, bone shape and soft tissue distribution differ significantly across users. Additionally, joint motion can lead to the sensing fabrics sliding subtly around the joint. Both individual differences and the motion artifact of the sensing fabrics pose challenges in achieving robust motion sensing in practice.

Applicants address the above challenges as follows. To disambiguate motion states, Applicants add a small piece of pressure fabric to augment the primary sensing fabric (i.e., strain sensors). The pressure fabric senses the pressure from the joint to the fabric during motions. Although it alone cannot provide precise sensing of subtle motion, it can steadily differentiate coarse motion states: flexion, extension, or the motionless state. Applicants characterize the strain fabric's resistance change in different motion states, and apply models from material sciences to compensate for the stress relaxation during the motionless state. Finally, Applicants leverage models in the literature of bio-mechanics to characterize the geometric relationship between skin deformation and joint angle. To deal with individual differences and motion artifact of the sensing fabrics, Applicants place two straps of strain fabrics as dual sensors above the joint. Applicants consider the differences of the stretch lengths of these two strain fabrics, which cancel out the impact of the body part thickness or slight offset of fabric positions.

Using the elbow joint as an example, Applicants fabricate prototypes in two sizes (27/20.5 cm lower-elbow perimeter, 31/23 cm upper-elbow perimeter) using off-the-shelf fabrics and micro-controller (Adafruit Flora). Applicants place strain fabrics and pressure fabrics in separate layers to enhance their sensing resilience and sensitivity. Inelastic fabrics are used as reference resistor for the micro-controller to monitor the resistance change of strain and pressure fabrics, where micro-controller and sensing fabrics are connected via conductive threads. Applicants tested the prototypes with ten participants to examine sensing accuracy and robustness. Applicants also sought participants' feedback on the wearing comfort. Applicants summarize various findings as follows, without limitation: (a) the system reconstructs elbow joint angles with a median error of 9.69° across users with different arm sizes, under motions with various speeds and magnitudes; (b) the sensing performance is robust against slight displacement (up to 1 cm) of the sensing fabrics around the joint, and the system allows gentle hand wash with minor degradation (7.64°) in sensing accuracy after wash; and (c) participants generally rate the prototypes comfortable to wear and flexible to adapt to various motions.

### Example 1.2. Background on Fabric Sensing

Fabric as a sensor presents numerous benefits, as it is soft, light, flexible, and thus easy to wear even during exercises. In particular, Applicants' Example considers off-the-shelf stretchable textile made of conductive threads as a primary sensing fabric. The sensing ability of such fabrics stems from their knitted micro-structures of the yarns, where an external strain causes changes in the number of contact points and contact pressure, resulting into changes in the contact resistance of the fabric. Applicants next experimentally examine the property of such fabrics and their sensing capability.

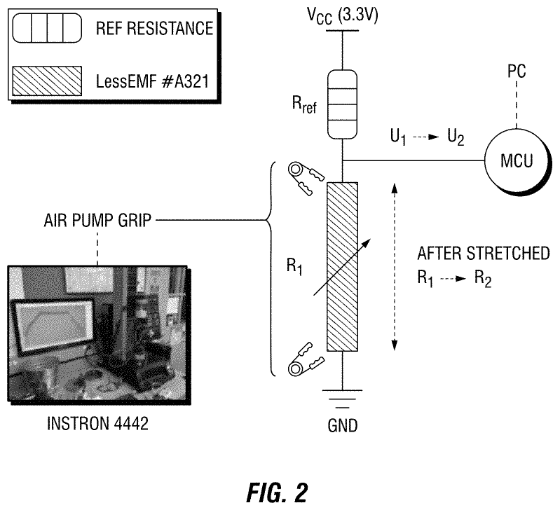

Experimental Validation. Applicants test an off-the-shelf stretchable conductive fabric (LessEMF #A321). With 76% Nylon and 24% elastic fiber, it can be stretched by up to 100% along its length/course direction and 60% along its width/wale direction. Its yarns are plated with a thin silver layer offering great conductivity. Applicants cascade the test fabric with a fixed-value reference resistance. Applicants use a micro-controller to measure the voltage on the test fabric to monitor its resistance change under various levels of strain. To systematically apply various levels of strain, Applicants use a professional tensile test machine (Instron 4442). FIG. 2 illustrates the overall setup. Applicants evaluate fabric's resistance change via the metric of resistance change ratio r_{ΔR}=(R₂−R₁)/R₁, where R₁ and R₂ denote the original and current resistance, respectively. With the original voltage as U₁ and current voltage as U₂, r_{ΔR} can be computed as:

$${r}_{Δ R} = \frac{{R}_{2} - {R}_{1}}{{R}_{1}} = \frac{{V}_{cc} {U}_{2} - {U}_{1} {U}_{2}}{{V}_{cc} {U}_{1} - {U}_{1} {U}_{2}} - 1 , \qquad ( 1 )$$

where V_{cc} is the power supply voltage from the micro-controller.

FIG. 3A plots the resistance change ratio as a 15 cm×3 cm fabric is stretched along its course direction. Applicants also plot the stress on the fabric recorded by Instron. Applicants obtain two observations. First, the fabric is very sensitive to motion, where even millimeter-level stretches cause noticeable changes in resistance (e.g., 2 cm stretch leads to 60% resistance change). It provides basis for sensing fine-grained motion. Second, as the strain grows, the change in fabric resistance is not monotonic; rather, it undergoes a sharp increase, followed by a gradual decline. Applicants observe similar patterns consistently across various test runs and across other types of stretchable fabrics Applicants have tested. This pattern can be explained by examining how contact points and contact pressure are altered under strain. Based on the Holm's theory, the contact resistance of a conductive fabric is inversely proportional to the number of contact points and the contact pressure. In the beginning when the fabric is being slightly stretched, the stress on the fabric is not evident and the change in the number of contact points dominates the resistance change. The low level of strain only causes fiber slipping within air gaps and forming tighter block structure. As a result, the number of contact points decreases, leading to an increase in contact resistance. As the fabric is further stretched under higher levels of strain, the stress on the fabric becomes the dominant factor that changes the resistance. Since there is no more space for yarns to form blocks, the number of contact points stays constant. The growing inner stress among yarns leads to the decline of the contact resistance. FIG. 3B illustrates fabric's micro-structure during different phases of the process.

Applicants further test the resistance change as Applicants stretch the fabric in the wale direction. Applicants cut two pieces of fabrics in the same size (15 cm×3 cm), one with course direction along its length and the other with wale direction along its length. Applicants stretch both fabrics to 30% of its original length and plot the resistance change ratios in FIG. 4A. Applicants observe that resistance change exhibits a similar trend yet with distinct details: stretch in the course direction leads to more significant changes in resistance (over 100%), while that in the wale direction leads to only around 5% change in the resistance. FIG. 4B and FIG. 4C show the microscope images of each fabric under 30% strain.

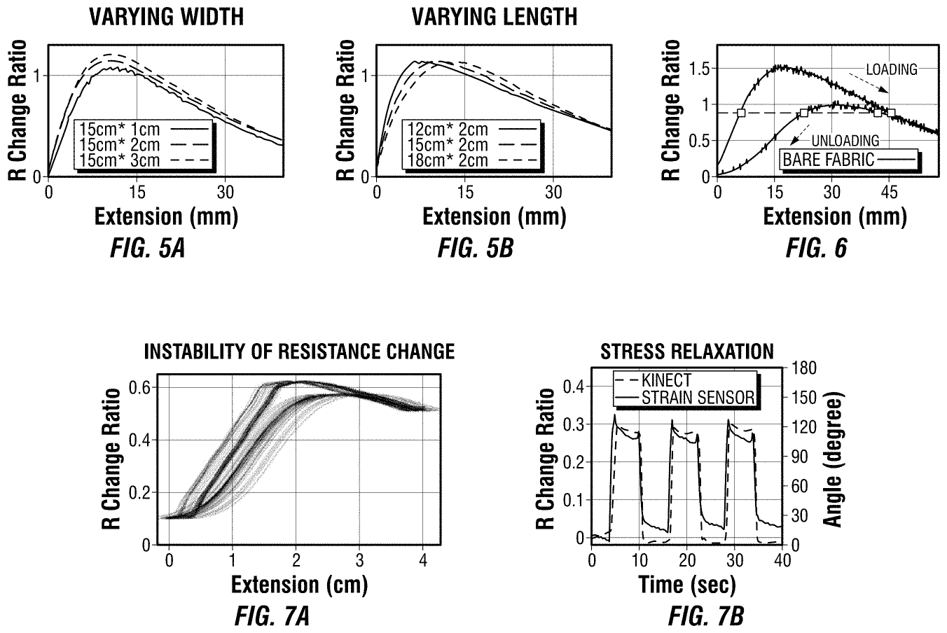

Applicants also examine the impact of fabric's size ratio on its sensitivity to strain. FIG. 5 compares various fabrics in various size configurations when they are stretched in the course direction. Applicants observe that increases in the width result into higher peaks, because more yarn blocks are formed, leading to fewer contact points and larger increase in resistance. On the other hand, under a fixed width, longer fabrics have more air gaps in the stretching direction and thus need more strain to reach the peak, leading to the peak occurred under larger stretch lengths. Additionally, Applicants observe that a thinner fabric (1-cm width) has more high-frequency noise in its signal response. Applicants hypothesize that the thinner fabric has weaker fiber strength, making it harder to keep a stable structure and stable resistance value. Overall, wider and longer fabrics are more preferable for sensing purposes.

Summary Applicants' experimental results validate that off-the-shelf stretchable conductive fabrics are sensitive to motion when they are stretched along the course direction. Even millimeter-level stretches lead to noticeable changes in resistance, providing basis for the fabric to sense skin deformation caused by subtle joint motion. Under increasing strain, the change in fabric's resistance is non-monotonic, starting with a sharp increase followed by a gradual decline. Wider and longer fabrics provide more stable, substantial change in resistance under strain.

### Example 1.3. Challenges in Fabric Sensing

While prior results are promising, fabric as a sensor also presents numerous challenges, especially for the purpose of accurately and robustly inferring joint angles. Applicants elaborate on the challenges as follows.

Elastic Hysteresis. The non-monotonic nature of the fabric resistance change under increasing strain indicates that there is no one-to-one mapping between the observed resistance change and the stretch length. Compounding this problem is the fact that the characterization of the resistance change also depends on the type of motion. When the fabric is being extended under strain (loading), its resistance change follows a curve that is different from that when the fabric is retracting as the strain is reduced (unloading). The difference is due to the energy dissipation caused by material internal friction. This phenomenon is referred to as hysteresis, an intrinsic property of elastic materials. As an example, FIG. 6 plots the ratio of resistance change during loading and unloading, with the LessEMF fabric (15.3 cm×3 cm in size). It implies that an observed value of resistance change can correspond to up to four possible stretch lengths, making it hard to uniquely infer the current skin deformation.

Instability. Furthermore, Applicants' experiments reveal that the impact of strain on fabric's resistance change slightly varies across repeated rounds of stretching. Thus, it is difficult to characterize the mapping between resistance change and extension length using a single function with fixed parameters. To examine this instability, Applicants stretch a 15 cm×1.5 cm fabric to 30% of its original length along the wale direction and repeat it for 160 rounds. FIG. 7A plots the resistance change ratio as the extension length increases. Applicants observe that curves from different rounds do not exactly overlap and exhibit slight offsets. A factor causing these offsets is electromagnetic (EM) coupling with ambient electromagnetic interference. Here each conductive thread of the fabric is similar to an antenna and the large number of threads can easily absorb ambient electromagnetic noise, affecting how fabric's resistance changes under strain.

Stress Relaxation. Another challenge comes from the fact that fabric resistance does not stay at a stable value under a constant strain. This is known as stress relaxation in the literature of material science, a property commonly seen in elastic polymer because of the loose connection of its fiber. FIG. 7B plots the resistance change ratio as a LessEMF fabric (15 cm×1.5 cm in size) is stretched three times by the Instron tensile machine, where each time the strain is kept constant for 60 seconds. Applicants observe that the resistance slowly drops by 12% after 5 seconds under a constant strain. Such instability brings more ambiguities in deriving a single deformation/stretch value based on the observed resistance change.

User Diversity and Motion Artifact. Finally, inferring the actual joint angle based on the sensed deformation on the skin is challenging given the differences of body parameters and motion patterns across users. This has been confirmed as one of the main sources of error in designing wearable systems in prior works. Specifically in Applicants' context, the relationship between the joint angle and the skin surface deformation depends on the bone shape and soft tissue distribution. Additionally, sensing fabrics can slightly slide during joint motions. As a result, the stretchable fabric can end up measuring skin deformation at various spots during the course of a motion, and the measurement inconsistency can hurt sensing accuracy. To address such motion artifact, prior works have considered applying a calibration in the beginning of each wearing, or the use of sensor array with machine learning algorithms. These methods, however, can handle only the initial offset caused by wearing, not the error during the motion. Applicants aim to seek solutions that are robust against slight position offsets of the sensing fabrics during joint motions.

### Example 1.4. System Design

Applicants address above challenges via four design elements: (1) Augmentation with Pressure Fabric: To tackle the lack of one-to-one mapping between stretch length and resistance change, Applicants augment strain fabrics with collocated pressure fabrics. Sensing the pressure from the joint motion, pressure fabrics are less sensitive to subtle joint motion and exhibit a narrower sensing range. However, they offer robust differentiation of coarse motion states (loading/extending, unloading/retracting, motionless, etc.). Once the motion state is identified, the system can select the proper modeling on strain fabric's resistance change for inferring the actual skin deformation; (2) Gaussian Distribution Curve: Instead of relying on a single function to characterize the resistance change ratio under varying stretch length, Applicants study the distribution of resistance change ratios to address the instability problem. Applying a statistical graphical method (Quantile-Quantile plot), Applicants validate that the distribution is Gaussian. Applicants then design a probability-based method to infer the extension length; (3) Modeling Stress Relaxation: Applicants model fabric's stress relaxation by leveraging the literature from material science. Applicants first apply the classical spring-dashpot model to characterize fabric's resistance change. Applicants then model the stress relaxation via a relaxation modulus and use the model to compensate for the resistance decline during stress relaxation; and (4) Dual Strain Fabrics for Inferring Joint Angle: Applicants derive the geometric relationship between skin deformation and joint rotational angle based on the anatomy of joints in biomechanics. Applicants propose the use of two parallel strain fabrics separated with a fixed interval and examine the difference between the two fabrics to cancel out the impact of fabrics' motion artifact and robustly infer joint angle.

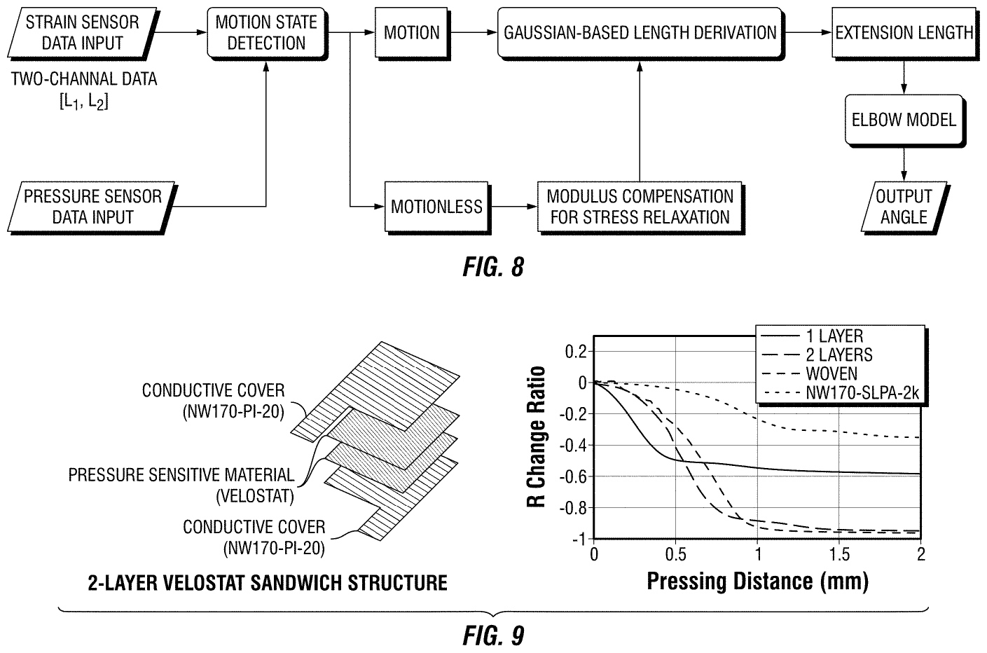

FIG. 8 illustrates the overall system flow. Each element is described in further detail below.

### Example 1.5 Augmentation with Pressure Fabric

Applicants first design element augments strain fabric sensing with pressure fabric to disambiguate motion states. Pressure is created by the squeezing of body joint and sensing fabrics. Pressure alone does not lead to precise sensing of joint motion. However, it can serve as an instructive reference to differentiate coarse motion states. Applicants consider collocating strain fabrics and a small piece of pressure fabric (actual placement is described in further detail below), so that they respond to the same joint motion and complement each other's sensing ability.

Characterization of Pressure Fabric. Pressure fabric commonly contains velostat, linqstat or other piezo-resistive materials whose resistance changes under pressure. Prior works have explored various layouts of such fabric, including grid, circle, and stripes. Pressure fabric is known to exhibit poor resilience and random signal drift because of its unsatisfactory mechanical property of the dielectric polymer layer. Thus, it is not suitable for accurately sensing subtle motion. However, its superior binary distinguishable upper and lower bounds with its linear slope enables it to be qualified motion monitor. Applicants place it on the top of the bulge formed by bending a joint, so that it differentiate coarse motion states such as flexion and extension.

Applicants have tested various materials (velostat vs. NW170-SLPA-2k by EeonTex), number of layers, and material of electrodes (woven conductive fabric vs. NW170-PI-20 by EeonTex) as the pressure fabric. The results of various configurations are shown in FIG. 9. Applicants observe that overall, resistance decreases when the fabric is pressed, leading to negative resistance change ratios. Resistance changes more significantly with more layers. Changing the conductive cover from NW170-PI-20 to woven conductive fabric results into noisier resistance values. The reason could be the porosity of woven fabric, which causes poor contact with the pressure sensitive material. NW170-SLPA-2k pressure sensitive fabric by EeonTex exhibits the smallest resistance change given the same level of pressure. Thus, Applicants settle on the two-layer structure with velostat in the middle as the pressure-sensing material.

Disambiguating Motion States. Based on pressure fabric's resistance change, Applicants aim to differentiate two motion states (loading, unloading) and a motionless state. Disambiguating these states is essential as each state corresponds to a different modeling of strain fabric's resistance change. Once a motion/motionless state is identified, the system can later apply the correct model on strain fabric's resistance change to derive the actual skin deformation (i.e., stretch length).

The differentiation of these motion states is based on the observation that joint flexion during loading raises the pressure sensed by the fabric (thus lowering the resistance), whereas joint extension during unloading lowers the pressure at the fabric (thus raising fabric resistance). Motionless states lead to relatively stable pressure at the fabric. Therefore, these motion/motionless states can be detected by examining the first-order derivatives of the pressure fabric's resistance change over time. A negative derivative indicates joint flexion/loading, while a positive derivative indicates joint extension/unloading. A derivative close to zero indicates motionless states.

Directly computing derivatives over pressure fabric's raw data, however, is prone to errors, given that sensor data are noisy and fluctuate over time. The noise can be introduced by the slight movement of the fabric over skin, or by the lack of sufficient force to the body. To deal with sensor noises and ensure a robust detection, Applicants smooth the raw data within a sliding window, interpolate data points between adjacent data points, and then compute the derivatives with the interpolated data points to obtain a more accurate trending of the raw data. Specifically, Applicants use Gaussian kernel fitting, an algorithm whose core is the widely-used Gaussian kernel.

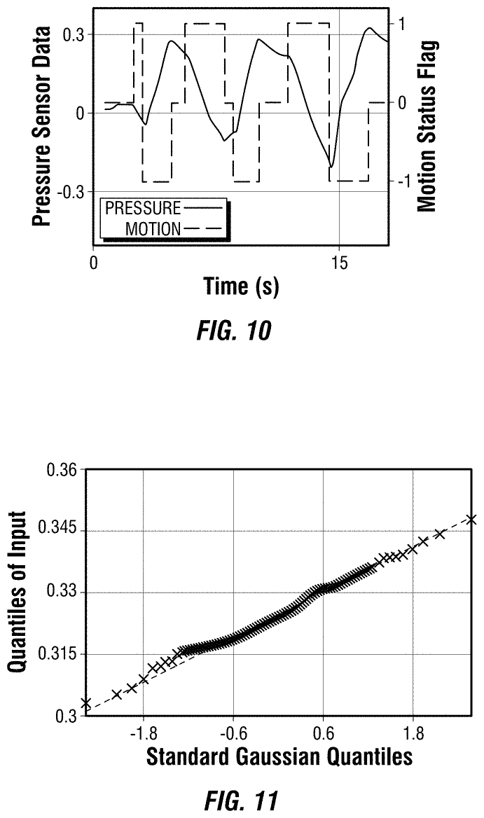

Algorithm 1 in Table 1 lists the detail in differentiating motion states, where \hat{x} denotes interpolated data points. Data from pressure fabric are first fed into a circular buffer where the pointer to the data moves circularly, so that Applicants can decrease the lag caused by real-time computation. Then Applicants smooth the data in the buffer within a sliding window avoiding mutation in sequential data stream. Next, for the newly updated buffer, Applicants interpolate data points based on the discrete data points in the buffer and then compute the first derivative with the interpolated data points. Applicants compare the derivative kₜ to a threshold k_{Motion} to determine if it is a motionless state. Applicants then examine the sign of kₜ to differentiate loading and unloading states. FIG. 10 shows an example run of the algorithm, where 1 means loading, −1 denotes unloading, and 0 means motionless states. For motionless status, pressure fabric data are further compared with two pre-set thresholds to decide whether the state is motionless with strain or without strain. With the identified motion state, next Applicants describe the modeling of strain fabric's resistance change to derive the stretch length (i.e., skin deformation).

TABLE 1
Description of Algorithm 1.
ALGORITHM 1: Detecting Motion States.

```
Input: Data from pressure fabric xₜ.
Output: Motion state: loading, unloading or motionless.
repeat
  Motion = Motionless;
  insert new data into the circular buffer bu f;
  for each bu f updated by new data xₜ do
    x̅ ← data interpolation;
    $\begin{matrix}Gaussian kernel ( φ ( \hat{x} , {x}_{t} ) = exp ( - \frac{{( \hat{x} - {x}_{t} )}^{2}}{2 {b}^{2}} ) ) regression on the \\ buffer bu f with kernel ’ s input scale b ;\end{matrix}$
    calculate the slope kₜ of current curve at data point xₜ;
    if |kₜ| < k_{Motion} then
      continue;
    else
      kₜ < 0 → Motion = Loading;
      kₜ > 0 → Motion = Unloading;
    end
  end
until no more data point comes in;
```

### Example 1.6 Derivation of Stretch Length

The second design element aims to infer stretch length based on fabric's resistance change. Given the instability of resistance change across repeated stretch cycles, Applicants examine the distribution of resistance change ratios under a given stretch length across different rounds of stretch. Based on the characteristics of the distribution, Applicants design a probability-based method to infer the stretch length, and next describe the analysis of the distribution and the probability-based method in detail.

Distribution Analysis. Earlier results indicate that the same level of strain (i.e., stretch length) can lead to different resistance change ratios across repeated stretches (FIG. 7A). Applicants set out to analyze the distribution of resistance change ratios under a given stretch length. Applicants stretch the fabric up to four centimeters 160 rounds on the tensile test machine while sampling fabric's resistance change ratio at 100 Hz. In total, Applicants obtained 400 data points in each round and 160 data points (i.e., resistance change ratios) for each stretch length. At certain stretch length, Applicants analyze the distribution of resistance change ratios. Using a statistical graphical method (Quantile-Quantile plot), Applicants' results reveal that the distribution is Gaussian. Quantile-Quantile (Q-Q) plot is a probability plot, which compares two probability distributions by plotting their quantiles against each other. Here Applicants fix one probability distribution as the standard Gaussian distribution and compare each distribution with it. FIG. 11 shows an example. Applicants observe that the points distribution is almost linear, suggesting that the distribution of resistance change ratios under a stretch length is Gaussian.

Probability-Based Length Derivation. Applicants exploit the Gaussian distribution of resistance change ratios for each stretch length to infer stretch length. Specifically, for each stretch length, Applicants derive the mean and variance of the Gaussian distribution of resistance change ratios and store them in a look-up table. Applicants then divide the look-up table to four classes, which are resistance increase during loading, resistance decrease during loading, resistance increase during unloading, and resistance decrease during unloading. Upon a sensed resistance change ratio, Applicants first determine the class it belongs to based on the slope-based method and the output of pressure sensor. Applicants then calculate its corresponding stretch length as well as probability based on each Gaussian distribution. The length with the maximal probability is selected as the inferred stretch length. Algorithm 2 lists the detailed steps.

TABLE 2
Description of Algorithm 2.

```
ALGORITHM 2: Extension length derivation.
Input: Data from strain fabric xₜ and motion state (Motion₁):
loading/unloading
Output: Extension length.
bu f : circular buffer;
repeat
  insert new data into the circular buffer;
  for each bu f updated by new data xₜ do
    slope-based method → Motion₂ = increasing/decreasing
    Combine the Motion₁ and Motion₂ → MotionClass;
    for each distribution in this class do
      calculate the probability Pᵢ
    end
    find the maximum probability Pₘₐₓ;
    return the extension length corresponding to Pₘₐₓ;
  end
until no more data point comes in;
```

Validation. To validate Applicants' length derivation algorithm, Applicants use leave-one-out cross validation to evaluate its accuracy. In each fixed extension length, Applicants have collected 160 resistance change ratios. Applicants use 159 values to calculate the mean and variance of one particular Gaussian. For 400 stretch lengths (i.e., Gaussian distributions), Applicants set the MotionClass of these 400 points and run Algorithm 2 to derive the stretch length. Applicants calculate the mean squared error (MSE) and root mean squared error (RMSE) for each extension length. Applicants summarize the results of two strain sensors in Table 3, respectively. Applicants observe that the algorithm's RMSE for each motion class is around 0.1 cm, which demonstrates that Applicants' algorithm performs well in mapping the resistance change ratios to extension lengths.

TABLE 3
Average MSE and RMSE of inferred extension based on
leave-one-out cross-validation for strain sensor 1 and 2.

| | Strain Sensor 1 | | Strain Sensor 2 | |
|---|---|---|---|---|
| State | MSE | RMSE/cm | MSE | RMSE/cm |
| Loading increase | 0.0063 | 0.0792 | 0.0045 | 0.0674 |
| Loading decrease | 0.0168 | 0.1292 | 0.0084 | 0.0918 |
| Unloading increase | 0.0231 | 0.1518 | 0.0211 | 0.1452 |
| Unloading decrease | 0.0204 | 0.1428 | 0.0046 | 0.0676 |

### Example 1.7. Modulus Compensation for Stress Relaxation

The third design element aims to compensate for stress relaxation. With the second design element, Applicants can derive the extension length in motion states. For motionless states where stress relaxation occurs, Applicants apply relaxation model from the literature of material science to compensate for the impact of stress relaxation. Its core lies in the characterization of strain fabric's resistance change.

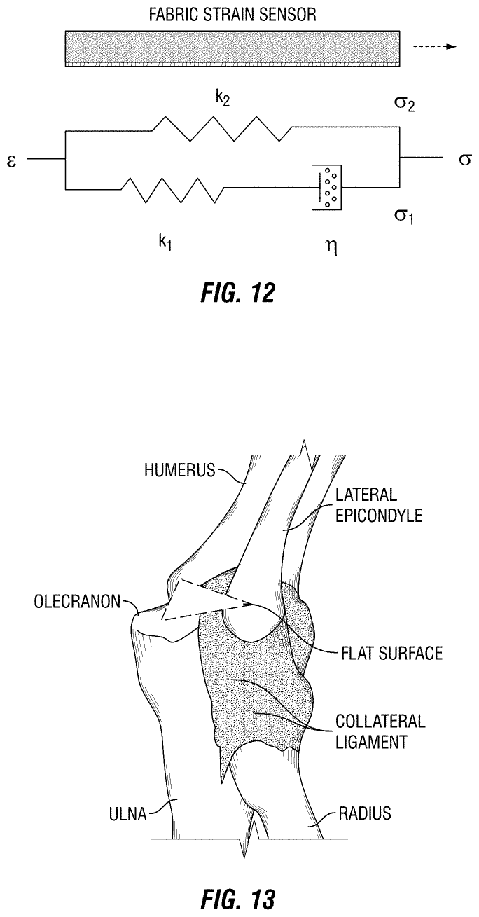

For most typical polymers whose conformational change is eventually limited by the network of entanglements or other types of junction points, a classical “spring-dashpot” model is effective to analyze its properties. Here, since Applicants add an additional layer of elastic material parallel to the sensing material, Applicants choose Maxwell form (instead of Kelvin-Voigt form) standard linear solid model to describe Applicants' strain fabric. As shown in FIG. 12, the model is composed of a spring-dashpot branch and another spring placed parallel with the branch. Applicants mark the stiffness of the whole system and two springs as k, k₁, and k₂, respectively, while that of dashpot as η. Applicants denote overall stress and the stress on each branch as σ, σ₁, and σ₂, respectively, and the deformation as ϵ, ϵ₁, and ϵ₂. Based on the physical properties of parallel springs, Applicants obtain these relationships k=k₁+k₂, σ=σ₁+σ₂, and ϵ=ϵ₁=ϵ₂.

According to the basic rule of spring and dashpot (σ₂=k₂ϵ₂ and σ₁=η·dϵ₁/dt), Applicants can derive the constitutive equation of this system as:

$$\frac{d σ}{dt} = \frac{d {σ}_{1}}{dt} + \frac{d {σ}_{2}}{dt} = \frac{d {σ}_{1}}{dt} + {k}_{2} \frac{d {ɛ}_{1}}{dt} = \frac{d {σ}_{1}}{dt} + \frac{1}{τ} {σ}_{1} , \qquad ( 2 )$$

where τ=η/k₂ is called the relaxation time constant.

In the stress relaxation state, the deformation value does not change, so dϵ/dt=0. Since σ=kϵ, Applicants have dσ/dt=k·dϵ/dt=0. By setting Equation (2) to 0, Applicants derive dσ/dt=−στ, assuming σ₁=σ₂=σ/2 to obtain a closed-form solution. Such constitutive equation links deformation to the stress on the material. By separating variables and integrating:

$${\int }_{{σ}_{0}}^{σ} \frac{d σ}{σ} = - \frac{1}{τ} {\int }_{0}^{t} d t , \qquad ( 3 )$$

is the initial stress and α is a constant.

Then the relaxation modulus Eᵣₑₗ(t) can be derived as:

$${E}_{r e l} ( t ) = \frac{σ ( t )}{{ɛ}_{0}} = {m}_{1} {e}^{- \frac{t}{τ}} + {m}_{2} , \qquad ( 4 )$$

where ϵ₀ is the initial deformation. Applicants fit the relaxation model with experimental data to calibrate parameters m₁, m₂ and τ. The three parameters of Applicants' relaxation model are: m₁=0.0233, m₂=−0.04591, τ=0.0289. SSE: 0.0795, RMSE: 0.01154.

### Example 1.8. Inference of Joint Angle

With the derived stretch length, the last design element is to infer the joint angle. Applicants analyze the anatomy of human joints to identify the relationship between the deformation and the rotation of joint bones. Applicants then discuss the use of dual strain fabric sensors to enhance sensing robustness.

Human joints can be categorized into six types: synovial joint, pivot joint, hinge joint, saddle joint, condyloid joint and ball and socket joint. Elbow joint is categorized as hinge joint, where the bones can only move along one axis to flex or extend. Applicants define the elbow joint angle as the angle between the current position of lower arm and the neutral anatomical position of it. Its moving range is roughly from −10° to 150°, and prior works from anatomy have confirmed the relationship between the skin surface deformation and elbow flexion angle. Specifically, three bones are involved in the rotation at the elbow joint: humerus on the upper arm, radius and ulna on the lower arm. As shown in FIG. 13, the distal end of the humerus and the proximal heads of the radius and ulna form a small flat triangle-like surface that is also supported by the collateral ligaments around the elbow. When people wave their arm, the ulna, with its upper end (called olecranon) will rotate around a virtual rotation axis, and as a result, the triangle-like surface will also rotate in a certain range. The rotation of this flat surface causes the deformation of skin around the joint.

Prior studies have proposed models to describe the relationship between skin deformation and joint rotation. The common problem of all these models is that they assume the virtual rotation axis of the ulna's rotation is known or easy to measure. However, in practice, it is hard to locate this axis because the axis is above the skin with an unknown height. Another problem of these models is that they assume the bulge (i.e., olecranon) of the joint is a perfect horizontal surface, while in fact it tilts with a slope that depends on the shape of the joint bones. To solve these problems, Applicants apply a general model based on anatomy that can cover different joint sizes. Applicants propose two parallel pieces of strain fabric to remove the need of locating the rotation axis.

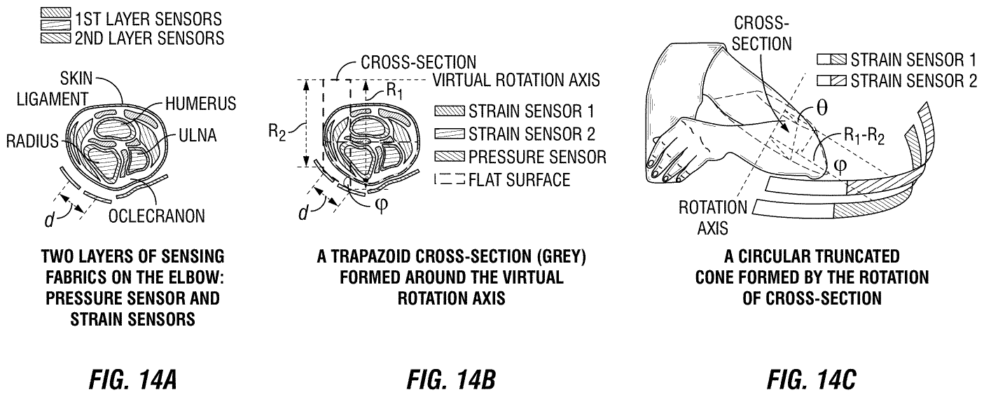

Sensor Layout. FIG. 14A and FIG. 14B show the placement of two strain fabrics together with the pressure fabric. Applicants adopt a two-layer structure to embed the two types of sensing fabrics: the first layer, which is closer to the skin, contains the pressure fabric that is responsible for determining the motion state, while the second layer, which is the outer layer, contains the two strain fabrics. FIG. 14B shows the cross-section (grey trapezoid) that rotates with elbow movement. Applicants mark the virtual rotation axis with a dotted line, whose location is unknown to the system. Based on the rotation axis, Applicants draw two parallel radiuses with two strain fabrics as the distal ends. Applicants denote the two radiuses as R₁ and R₂, and the distance between them is d. Applicants denote the inclination angle of flat triangle surface (marked in FIG. 13) as φ. R₁ and R₂ are unknown, while d and φ are body parameters that Applicants can measure during calibration.

Dual Input Sensing. FIG. 14C illustrates how Applicants infer the movement angle with all the parameters Applicants set in the previous step. The extension on the two parallel strain sensors can be seen as two arcs of one virtual circle (whose center is the virtual axis). Applicants denote the length change of each arc as L₁ and L₂ (i.e., stretch lengths of the two strain fabrics) and the rotational angle (supplementary angle of joint angle) as θ. Note that if Applicants view from the projection direction (as shown in FIG. 14C), the rotation of the cross-section will become a circular truncated cone. With all these parameters, the rotational angle θ can be computed as:

$$θ = \frac{{L}_{1} - {L}_{2}}{{R}_{1} - {R}_{2}} = \frac{{L}_{1} - {L}_{2}}{d cos ( ϕ )} , \qquad ( 5 )$$

where d and φ are calibrated parameters, L₁ and L₂ are the stretch lengths sensed by the two strain fabrics.

Thus, Applicants avoid the potential error imported from inaccurate estimation of rotation axis and the radiuses (R₁, R₂). Furthermore, the use of two pieces of strain fabric also allows the system to be robust against small position offsets of these strain fabrics during joint motion. Since the derivation of rotational angle in Equation (5) uses the difference of two stretch lengths, small position offset of the strain fabrics is canceled out, given that the two strain fabrics are collocated on roughly the same slope of the skin.

Applicants calibrate parameters d and φ based on a user's bone shape and tissue distribution. First, depending on user's joint size and thickness, the distance d between the two strain fabrics can be slightly extended as a user puts on the prototype around a joint. The actual value of d can be measured during a user's first wear of the prototype. Second, φ, the slope of the flat surface around a joint, can differ across users. Since it is not easy to directly measure it on the skin surface, Applicants calibrate it by asking the user to perform a full flexion of a joint, i.e., transitioning from full extension to full flexion. Given the maximal rotational angle θ* of a joint (e.g., the maximal rotation of an elbow joint is around 150°), the angle φ can be computed as φ=arccos((L₁′−L₂′)/dθ*) based on Equation (5), where L₁′, L₂′ are the stretch lengths of the two strain fabrics during the full flexion. For both parameters (d and φ), their values stay relatively constant for a given user. Thus, the system requires only a one-time calibration for a user, instead of a calibration for every wearing instance even with the same user, entailing a lower calibration overhead than prior works.

### Example 1.9. Prototype Implementation

Applicants build prototypes in two sizes: (1) 27 cm and 31 cm as the lower-elbow and upper-elbow diameter respectively; and (2) 20.5 cm and 23 cm as the lower-elbow and upper-elbow diameter, respectively. Each prototype is composed of stretchable conductive fabric as strain sensors, inelastic fabric as reference resistor, pressure-sensitive conductive sheet as pressure sensor, stainless steel conductive thread as wires, micro-flex compression knit fabric as layers, sewable snaps and iron-on adhesive hem to connect, sports kinesiology tape to insulate, and a micro-controller (Adafruit Flora). Instead of inventing new high-cost materials, all the components in the system are easy to obtain and at low prices (<$20 except the micro-controller). While aiming to optimize the performance, Applicants also provide for a device that is comfortable to wear and washable.

### Example 1.10. Sensing Fabrics

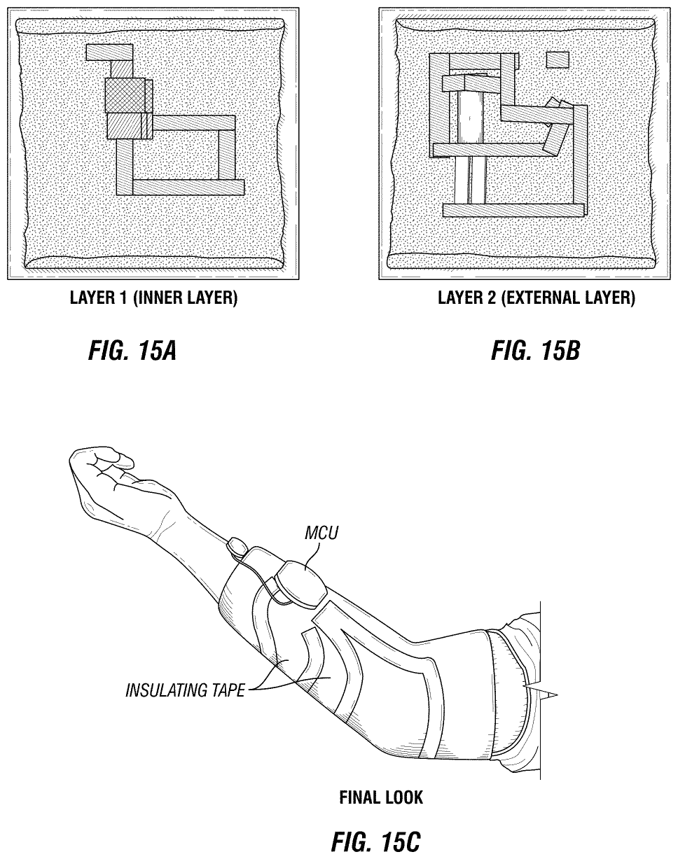

Layers. Applicants cut two pieces of non-conductive micro-flex knit fabrics as the bases of two layers. As shown in FIG. 15A, in Layer 1, the pressure fabric is placed in the inner layer close to the skin so that its resilience can be supported by the outer layer, while in Layer 2, the strain fabrics are placed in the outer layer for the maximal degree of extension as in FIG. 15B. This two-layer design structure has two advantages: (1) it provides physical isolation between pressure sensor and strain sensors, so that the deformation on the fabric strain sensors will not be limited by the inelastic pressure sensor; and (2) borrowing idea from PCB manufacture, such double-layer structure can spare more room for the conductive thread to connect different parts because the thread has access to two surfaces now with the help of some connecting holes. For the first advantage, Applicants' early-stage prototypes confirmed that, if Applicants put the sensors in the same layer, the closest strain sensors to the pressure sensor can hardly respond to strain because of the shared gap with pressure sensor, though the gap itself is flexible. For advantage two, such design makes the implementation of prototype much easier and also it reveals stronger anti-interference ability than one-layer version, since there is no cross or squeeze of conductive thread on the surface even if with vigorous movement. The sensing fabrics on two layers are totally independent and well-insulated from each other. The only connection between the two layers are three conductive sewable snaps so that the circuit on Layer 1 also gets the power supply and transmits voltage value to the micro-controller.

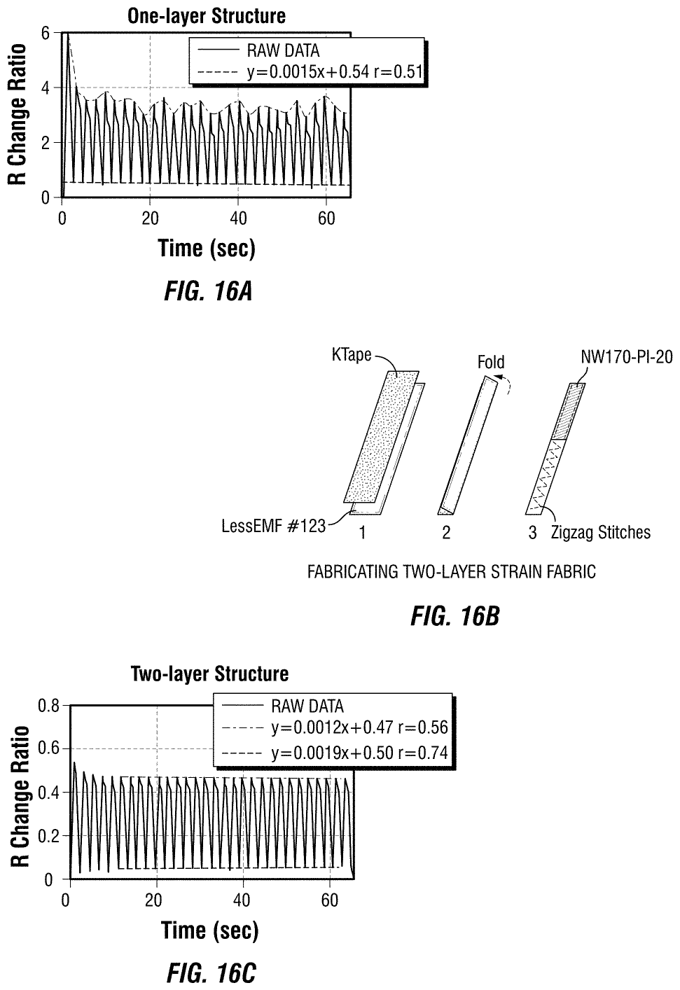

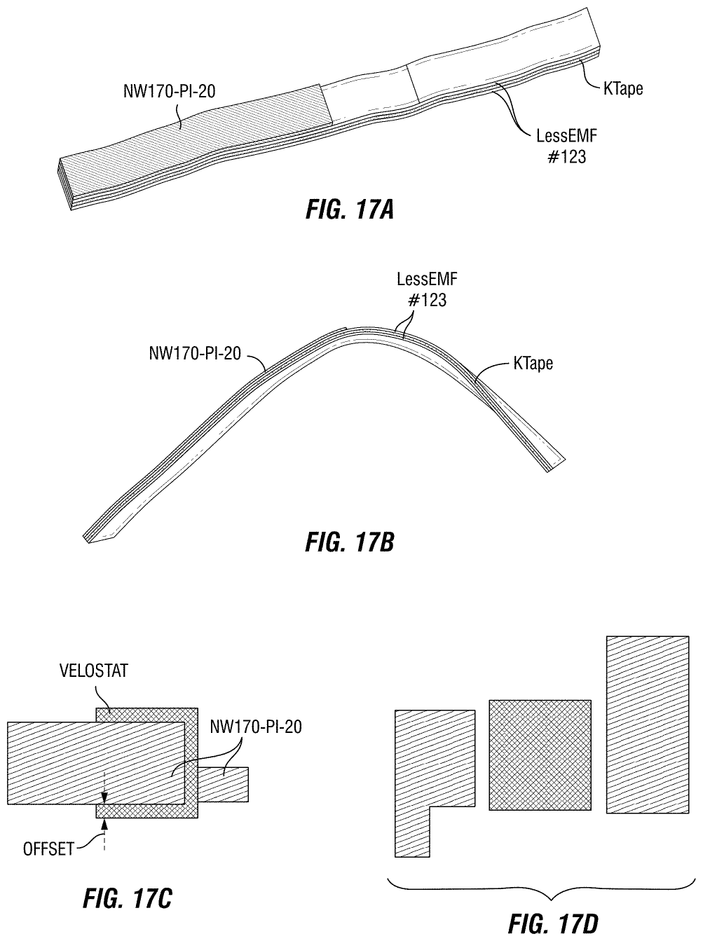

Strain Fabrics. Applicants use LessEMF #A321 as the stretchable conductive fabric, which is silver plated with 76% Nylon and 24% elastic fiber fabric. Applicants' experiments show that a single-layer strain fabric does not offer mechanical stability after repeated stretches, leading to large fluctuating peaks of resistance change ratios over time (FIG. 16A). To address this issue, Applicants design a two-layer structure, where Applicants paste a layer of insulative elastic tape (KTAPE) atop the LessEMF fabric, fold it with KTAPE in the middle and LessEMF on the top and bottom, and then sew these layers with elastic zigzag stitches. The strain sensing fabric is cascaded with an inelastic conductive fabric (EeonTex NM170-PI-20) serving as the reference resistance. The reference fabric has a similar two-layer structure and layers are sewed using simple inelastic stitches (FIG. 17A and FIG. 17B). Applicants do not choose an electrical resistor (e.g., mm-scale surface mount device (SMD) resistor) as the reference resistance mainly because connecting a small SMD resistor to the sensing fabric requires soldering the PIN of the resistor to conductive threads. Applicants' experiments show that such soldering introduces much higher noise than that of connecting fabrics via conductive threads. However, Applicants do not rule out SMD resistors as an option.

FIG. 16B illustrates the fabrication process. Such a two-layer structure improves the stability of output voltage (FIG. 16C). Specifically, strain fabrics (LessEMF) are cut into pieces of 15 cm×3 cm in size, resulting in a 1.5 cm width sensing fabric after the folding. It is then connected to a 7 cm×1.5 cm static conductive fabric (EeonTex NM170-PI-20) as the reference resistance. Applicants sew two strain sensors onto Layer 1 using zigzag stitches, which do not influence the elasticity of sensors. They are the main sensing fabrics in the prototype and are placed roughly in accordance with the flat surface of the elbow.

Pressure Fabrics. As mentioned before, Applicants use 2-layer velostat sandwich structure for the fabric pressure sensor (FIG. 17C). Applicants place a 3 cm×4 cm piece of static conductive fabric at the bottom, another 3 cm×7 cm one on the top and two 3 cm×4 cm pieces of 1 mm-thick pressure-sensitive conductive sheet in between (FIG. 17D). Both the top and bottom layers perform as reference resistors and have an extra part as pins to connect with conductive threads. Applicants use iron-on adhesive hem to stick them together and cover the sensor with kinesiology tape to fasten and insulate. As pressure that elbow exerts on the sensor without any supporter contributes more to spatial deformation rather than forming a sagging area, Applicants sew a piece of stretched highly-elastic fabric outside the sensor unit to provide a reversed pressure while bending arms. The pressure sensor is laid on Layer 2 beside two strain sensors on Layer 1 and exactly covers the olecranon part of elbow (see above). The final look of the prototype is shown in FIG. 15C.

### Example 1.11. Computing Unit

Applicants use an Adafruit FLORA v2 board whose core board is ATmega 32u4 to digitize analog voltage signals of each sensor. FLORA is a small (1.75 in diameter, weighing 4.4 grams) and fabric friendly board with sewing tap pads, which interferes little with motions and can be sewed on the sleeves. ATmega 32u4 is an 8-bit micro-controller with 32K bytes of ISP Flash. During signal digitization, Applicants use 250 kHz ADC rate, which is sufficient in capturing low-frequency motions. Timer frequency is 100 Hz and results are stored in the MCU. Applicants sew eight conductive snaps on the layers, among which three electrically connect two layers and five attach the sewing tap pads on Flora to Layer 1 so that the micro-controller is detachable. Also, Applicants sew conductive threads into the layers as leads to connect three sensors with the micro-controller.

### Example 1.12. Points for Attention in Fabrication Process

Applicants summarize a few points for attention on the prototype fabrication. First, when Applicants use conductive threads as the leads to connect the pressure sensor with the snaps, the intersection between the threads and the sensor requires a piece of conductive elastic fabric. This is because the pressure fabric is inelastic, which will leave more gaps between the threads and the fabric, causing sharp increases in resistance. Since Applicants rely on resistance change ratio to determine the loading or unloading state, a large resistance value will lead to low sensitivity to small resistance change. Another suggestion for sewing is to place the conductive thread in the bottom bobbin, given that the conductive thread is too thick to go through the hole of the sewing machine. Finally, with the two-layer design, the outer layer can also possibly contact human skin, which would introduce signal interference. To physically isolate layers and shield against unwanted touch, Applicants cover all the conductive threads with KTAPE.

Although Applicants' current fabrication process is lengthy with manual efforts, most of the time-consuming steps (e.g., cutting, sewing, folding) can potentially be automated to scale up the fabrication process. The exact placement of various sensing fabrics during the assembling is based on the typical size of the bulge of human elbow joint, and thus can be realized as a repeatable process. The automation of fabrication process is described in further detail below.

### Example 1.13. Evaluation

Applicants have recruited 10 participants from Applicants' local institution to evaluate the prototype. The detailed user information is summarized in Table 4 and Table 3. In this section, Applicants first describe the experiment setup and the method of collecting the ground truth. Then the system accuracy is demonstrated given various movement patterns. Applicants further examine the reliability of the system regarding the sensor movement and washability. Finally, Applicants examine the comfort level of the prototype via a user study.

TABLE 4
Information of ten participants in the evaluation.

| | Gender | | Height/cm | | | Weight/kg | | |
|---|---|---|---|---|---|---|---|---|
| | Male | Female | <170 | 170-180 | >180 | <60 | 60-80 | >80 |
| # of Users | 7 | 3 | 2 | 4 | 4 | 3 | 4 | 3 |

TABLE 3
The upper and lower arm size of each participant.

| User ID | 1 | 2 | 3 | 4 | 5 | 6 | 7 | 8 | 9 | 10 |
|---|---|---|---|---|---|---|---|---|---|---|
| Upper | 31 | 29.5 | 30 | 32 | 27.5 | 23 | 24.5 | 22.5 | 23 | 23 |
| Arm | | | | | | | | | | |
| Size | | | | | | | | | | |
| (cm) | | | | | | | | | | |
| Lower | 27 | 22.5 | 23.5 | 24 | 24.5 | 21.5 | 25 | 23 | 20.5 | 22 |
| Arm | | | | | | | | | | |
| Size | | | | | | | | | | |
| (cm) | | | | | | | | | | |

Setup. Each participant is instructed to wear the prototype with the size that fits his/her arm. With two available sizes of the prototype (see above), participants whose upper arms are with perimeters larger than 24 cm wear the larger size. Various types of motion patterns are designed and demonstrated in videos. After watching the video, participants imitate the motion, while the prototype records fabric sensing data, which are then transmitted to a laptop running the joint angle inference algorithm. In the end of the experiment, each participant fills in a questionnaire to rate the level of tightness, comfort and flexibility of wearing the fabric prototype, as well as their feedback on the one-time calibration. Additional feedback is also solicited on the comparison of the prototype and commercial compression sleeves.

To collect ground truth data, Applicants choose the VICON system because of its higher accuracy in comparison to other motion-capture systems such as Kinect. Its sensing ability relies on the reflective skin markers and infrared cameras and the system error is less than 2 mm. Applicants attach optical markers to each participant's elbow to measure the skin deformation and elbow angle. Applicants' early tests reveal that with only three markers attached to the arm (on upper arm, elbow joint and lower arm respectively), VICON occasionally loses track of the markers as these small markers can be easily occluded. To address this problem, Applicants create two rigid planes with hard paper board and place them onto the upper and lower arm skin surface. These planes reduce occlusions and help VICON track the arms and output the joint angle even if one or two points on the plane are missing. Meanwhile, to synchronize and compare data from VICON and Applicants' system in real time, Applicants develop a cross-platform tool programmed in C to gather data from MCU and VICON system together. Applicants integrate the calibration procedure into the tool. The experiment with each participant takes roughly 40 minutes, where calibration and adjustment takes only three minutes.

### Example 1.14. Accuracy of Inferring Joint Angles

Applicants start by examining the accuracy of sensing joint angles using Applicants' fabric prototypes. Specifically, the following sequence of motions is designed for each participant: (1) flex the elbow by various degrees (i.e., 45°, 90°, and 150°), and repeat the motion at three levels of speed. Repeat each motion three times and then rest 30 seconds in the end; (2) repeat the above motion sequence at the same three levels of speed but pause 5 seconds at 45°, 90°, and 150°. Rest 30 seconds after this step; and (3) perform free-form motions, including waving tennis racket, performing the yoga tree pose and belly button challenge.

The first two steps are controlled motions for evaluating the efficacy of various design elements to handle different movement angles and speed. Specifically, by adding the 5-second pause, step (2) is meant to examine the efficacy of the design element on stress relaxation and drift compensation (see above). The third step evaluates the system under free-form motions.

Applicants next discuss results, starting from overall accuracy and then diving into the analysis on controlled and free-form motions. Applicants evaluate accuracy by calculating absolute joint angular errors, i.e., the absolute differences between inferred and ground-truth joint angles.

### Example 1.15. Overall Accuracy

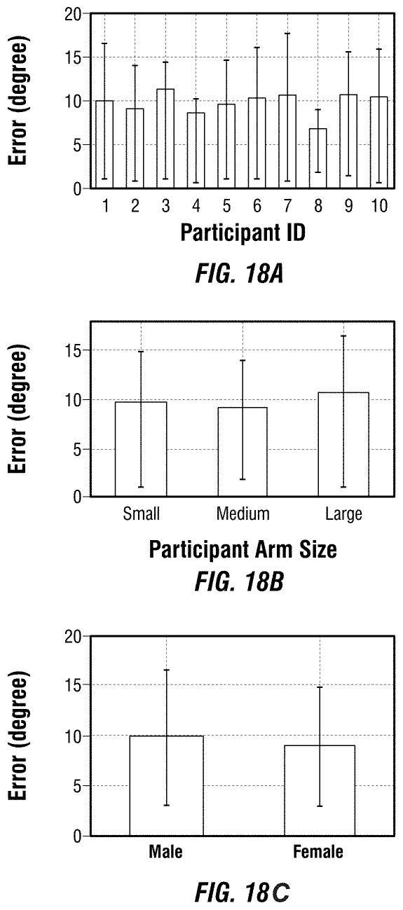

Applicants evaluate system's overall accuracy by aggregating joint angular errors of all participants in performing all steps of the motion sequence. FIG. 18A plots the average angular error for each participant, where error bars represent the range between the 5-th and 95-th percentiles. Overall, the median accuracy across users is 9.69°, which can facilitate rehabilitation applications that aim to limit the range for patient's joint movement (e.g., from 30°-100°). Comparing results across participants, Applicants observe that the average angular error with participant 8 is the lowest. This is because the arm size of this participant is used as the reference for building the fabric prototype worn by this participant. The prototype best fits this participant.

Applicants further analyze the impact of participant's arm size on joint angular error. Applicants divide participants into three groups based on the perimeters of their upper arms: small (upper arm perimeter <24 cm), medium (24 cm≤upper arm perimeter ≤28 cm), and large (upper arm perimeter >28 cm). Applicants focus on the size of the upper arm because lower arm size has almost no impact on wearing tightness. Applicants aggregate joint angular errors of participants in each group, and plot the average as well as the 5-th and 95-th percentiles for each group in FIG. 18B. Applicants observe that for participants with medium arm size, their average joint angular error is the lowest. This is because the prototypes best fit these participants without being too loose or tight. Too tight contact between skin and fabric sensor limits the spatial deformation of velostat material, which causes errors in motion state determination. On the other hand, when the prototype is too loose, the fabric fails to capture minor skin deformation, which also leads to larger errors.

Applicants also examine the overall accuracy for male and female participants. FIG. 18C plots the average with error bars covering the 5-th and 95-th percentiles. Applicants do not observe differences with statistical significance. This is expected as their arms have the same anatomic structure.

### Example 1.16. Controlled Motion

Applicants now zoom in on results of controlled motions (step (1) and (2)), aiming to examine the impact of pause, motion speed, and flexion angle on the accuracy of sensing joint angles.

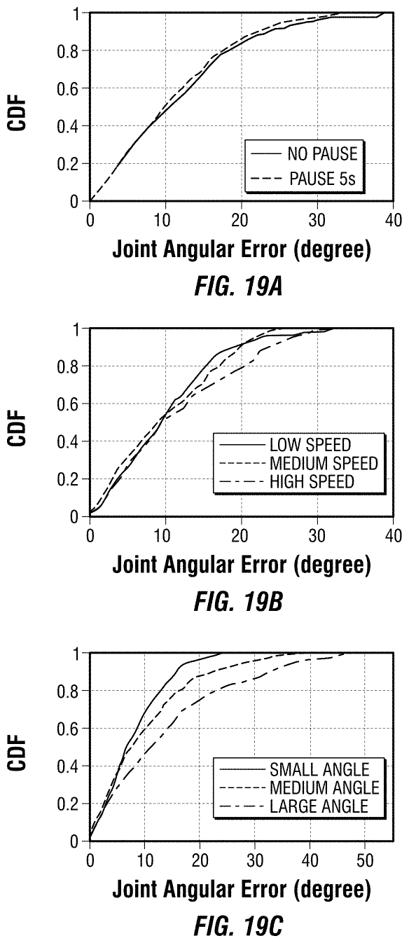

Pause. To analyze the impact of pause at different stress levels, Applicants aggregate the angular errors in step (1), where there is no pause between joint movements. Applicants compare them to that in step (2), where a 5-second pause is added. FIG. 19A compares the cumulative distribution functions (CDFs). Applicants observe negligible differences between the distributions. It indicates that the system can track continuous joint motion while controlling the error accumulation, thanks to the design element on stress relaxation and drift compensation (see above) that deals with the drift error. However, the distribution of errors during continuous motion has a slightly longer tail, with the maximal error of 38.7°, compared to the maximal error of 34.3° during motions with 5-second pause. Applicants hypothesize that the longer tail is due to the extra arm vibration that seemingly occurs during continuous motions. In the experiments, Applicants observe when participants continuously flex their elbows, their arms slightly shake in the end of each flexion. The shaking results in vibration of fabric strain sensors and occasion peaks in angular errors.

Speed. Speed is another factor that may affect sensing accuracy. Applicants evaluate the prototype with three levels of speed: low speed (20°/s), medium speed (50°/s), and high speed (80°/s). As shown in FIG. 19B, higher speed causes larger errors, while for the low speed, 80% of errors are within 15°. Given that the modulus Applicants use for stress relaxation and drift is time-dependent, it is likely that the relaxation is loosely related to speed. Another possible reason is that for higher speed, the fixed window size (12) used by the motion detection algorithm (Algorithm 1) may be not wide enough for the regression kernel to reconstruct the curve. The confused motion detector may output some unreliable motion status at high speed. Applicants envision a window-size selection module that adapts the window size to the current speed. It is challenging since practical movement is not controlled and speed can vary every moment. Applicants can seek methods to estimate speed in real time so that the window size can be adapted properly.

Flexion Angle. Applicants also examine whether the actual flexion angle affects the resulting joint angular error. Applicants divide angular errors into three groups based on the actual flexion angle: small (0°-45°), medium (45°-90°) and large (90°-150°). FIG. 19C compares the accuracy results for motions with different flexion angles. Applicants observe that larger flexion angles lead to slightly larger angular errors. Specifically, the median error under small flexion angles is 9.71° with 31.03° as the 95-th percentile, which increases to 13.24° with 37.21° at its 95-th percentile under large flexion angles. Applicants hypothesize that this trend is due to the influence of deformation in wale direction. When the flexion angle exceeds 90°, the contact of the upper and lower arm forces the strain sensor to extend in the wale direction. Given that resistance change along fabric's wale direction is less sensitive to the extension (see above), it is difficult to capture small extensions, resulting into larger errors. To mitigate this problem, Applicants envision methods to capture extension in the wale direction.

### Example 1.17. Free-Form Motion

Applicants further examine sensing accuracy under free-form motions. Specifically, Applicants consider the following motions: (1) waving a tennis racket several times, to check the performance of the system at high speed and in wide range of elbow flexion; (2) tree pose from yoga, to examine the ability of reconstructing motionless and steady gesture; and (3) belly button challenge, a popular movement among young people to test body flexibility, which requires using one hand to touch one's belly button from the other side across the back. Applicants expect to see similar performance when the elbow movement is done with some full-body motion.

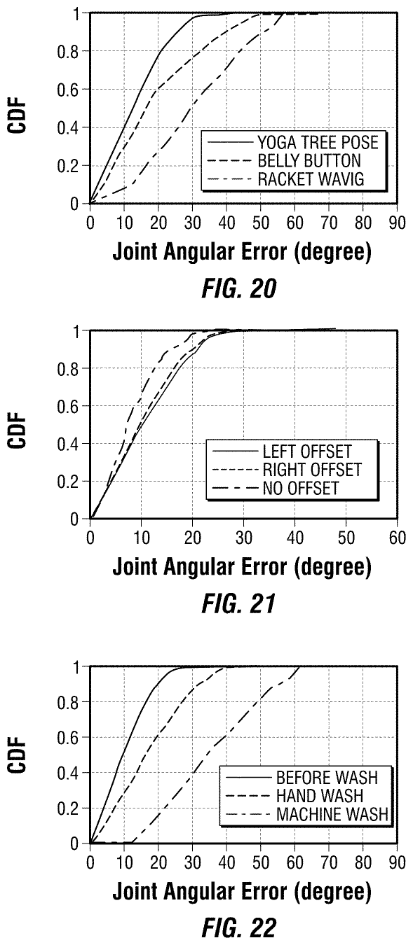

FIG. 20 plots joint angular errors for the above three motions. Applicants obtain three observations. First, slow movements (e.g., tree pose, belly button) in general achieve higher accuracy with the prototype. This is because of the slow restoration procedure of the fabric under sudden strain, which introduces larger errors when tracking quick motions. Second, belly button motion leads to higher errors than the tree pose. Applicants hypothesize that it is due to the rotation of lower arm and wrist during this motion. Since the prototype is currently designed to track mainly elbow flexion, joint rotation in other degrees of freedom introduces extra deformation and pressure and can interfere with the sensing of joint angle. Finally, similarly to prior results on speed and flexion angle, motions (racket waving) with larger flexion angles and higher speeds lead to higher errors. During these motions, Applicants observe more wrinkles accumulated around the fabric of the elbow region. Wrinkles affect fabric resistance and cause larger errors in inferring joint angles.

### Example 1.18. Sensing Robustness

After examining the sensing accuracy, Applicants now move on to evaluating the sensing robustness of the fabric prototypes. Applicants focus on two practical factors that can affect fabric's sensing performance: 1) the displacement of sensing fabric around the joint region, which can be caused either by initial wearing or by later motions; and 2) repeated wash of the fabric prototype after the MCU is detached.

Fabric Displacement. The prototype performs the best when the sensing fabrics are placed in the right region of the elbow: the two strain fabrics are right around the ulna bone while the pressure fabric is right around the radius bone (FIG. 14). In practice, however, these sensing fabrics can slightly shift away from their best sensing spots, either because of continuous joint motion or the improper alignment during the initial wearing. To evaluate the impact of fabric displacement, Applicants instruct one participant to wear the prototype and flex the elbow by various angles (45°, 90°, and 150°) with a speed of 20°/s. Applicants emulate fabric displacement by slightly moving the sensing fabrics from their optimal sensing spots by 1 cm, either to the left or to the right. Applicants then compare the results to that without any offset to examine the impact of fabric displacement on sensing accuracy.

FIG. 21 plots the CDFs of angular errors with and without fabric displacement. As expected, offsets from the center/optimal position slightly increase the joint angular error. The main reason is that these small offsets cause one of the strain fabrics to move beyond the olecranon, which breaks the parallelism of the two fabrics. This introduces error in calculating the angle from the differential inputs. Displacement with the right-side offset leads to slightly smaller errors because the pressure fabric is on the left of the strain fabrics. As a result, moving to the right has less influence on the pressure fabric. Note that in practice, larger displacement can be easily noticed and corrected by simply placing the fabrics to the center of the elbow. Overall, the results reveal that small offsets (≤1 cm) introduce a minor increase in joint angular errors, demonstrating the efficacy of the dual input sensing (see above) in dealing with motion artifact.

Washability. Washability is a useful parameter for long-term uses of the fabric prototype. The only electrical component of Applicants' prototype is MCU, which can be easily detached through a button (see above), leaving the rest of the prototype washable. To examine the impact of wash on the fabric's sensing ability, Applicants test two types of washing: machine wash and hand hash, where the latter is more gentle. Applicants instruct a participant to wear the prototype before and after washing to perform all steps of motion sequence and compare the errors in inferring joint angles.

FIG. 22 compares the CDFs of joint angular errors before and after wash. Applicants observe that both types of wash degrade fabric's sensing performance. This is partially because fabrics shrink to some extent after wash, even if Applicants dry the prototype thoroughly in open air. In comparison, machine wash affects the performance more severely, resulting into a median angular error of 34.1° and a maximum error of 72.4°. This is mainly because after machine wash, some conductive threads are broken while others have loose contact with the fabric. Both greatly affect the sensing of resistance change ratios. Gentle hand wash, on the other hand, can better protect the conductive threads and cause minor degradation (7.64°) to the sensing performance. Applicants envision further improving the prototype's robustness against wash through better fabrication.

### Example 1.19. Comfort Level

To evaluate the comfort level of Applicants' prototypes, Applicants conduct a user study with ten participants to solicit their input. All participants of Applicants' experiments are asked to fill in a questionnaire. The questionnaire asks participants to rate with the scale of 1-9 the following attributes of the prototype system: (1) tightness (1—too tight, painful to wear; 9—very loose, do not feel wearing it at all), (2) flexibility (1—too rigid, cannot perform any motion; 9—very flexible, no hindrance to performing any activities), (3) one-time calibration in the beginning (1—too long, tedious; 9—quick, not an overhead at all), and (4) the overall comfort level of wearing the prototype (1—very uncomfortable; 9—very comfortable, do not mind wearing it at all times).

Applicants calculate the average ratings of the above four attributes, and they are 6.1, 8.5, 8.3 and 8.8 respectively. It demonstrates that the prototype achieves an acceptable comfort level: the sizes of the prototype adapt well to target users with different figures; the prototype does not hinder motions for sports and everyday use; one-time calibration after purchasing it is relatively quick and acceptable. Additionally, 9 participants think applying the system into the teaching process of certain sports like yoga or gymnastics would be very helpful (8 points or more) to their progress.

Additionally, Applicants have also solicited participant feedback comparing the comfort level of the prototype with other commercial sleeves. Applicants invite participants to wear off-the-shelf compression sleeves for 30 minutes, which roughly the same duration of wearing the fabric prototype during Applicants' experiment. Overall, participants have not mentioned any issues related to wear comfort using the prototype. A participant (P5) comments that, “There is no difference between wearing your prototype and the compression sleeves.” Another participant (P2) comments that, “When you are playing sports, this would not have any influence just as the protection arm sleeves, while it tightness might make you feel a little bit uncomfortable when you are typing before a computer.”

### Example 1.20. Related Work

Applicants divide existing works on fabric sensing into two main categories based on their sensing capabilities.

Motion Classification. The first category of works focus on differentiating a pre-defined set of motions or activities. In particular, two types of resistive fabric strain sensors have been explored in the past. The first type is made of textile structure with conductive yarns. The embedded conductive yarns form many conductive paths, where contact resistance would change following the given mechanical stretch. Other studies chose to use silver yarn and nylon as the material and produced a weft-knitted strain sensor. Others created knitted strain sensors by using stainless steel and carbon yearns. The advantage of this kind of strain sensor is that the integration of the conductive medium into the textile structure is easily accomplished by slightly modifying existing knitting machine procedure. However, the mixture or the filling material of the sensing yarn is always rigid, which may cause discomfort while wearing it.

The other type is to coat elastic material (e.g., lycra) by intrinsically conductive polymer (ICP). A PPy-coated Lycra fabric has been developed and presented a wearable system able to reveal the status of body kinematic chains. Other studies coated PPy on Nylon-spandex (95:5) and obtained a strain sensor which can keep sensing until 60% extension. Others have used off-the-shelf PPy-coated fabric LTT-SLPA (28% Elastane, 72% Nylon) made by EEONYX Inc. to develop a deformable musical keyboard. This suggested that the maximum strain the sensor could handle is around 100-150%, which indicates this fabric is ideal for wearable strain sensor as its working space is large enough for skin surface deformation happening on the body.

Applicants are inspired by these prior works on fabric sensing. However, Applicants' work differs in that Applicants' system achieves much higher sensing granularity by enabling the inference of actual joint angles, rather than differentiating pre-defined gestures or poses.

Motion Reconstruction. The second category of works aim to compute joint angles. Most of these works rely on specially-designed materials. As examples, some have used a novel thermoplastic elastomer strain sensor and attached it to the back of a tight-fitting cloth to recognize upper body postures. Others have poured liquid silicone on a three dimensional (3D) print mold and inject EGaIn to generate a soft strain sensor. While others chose graphene, a special carbon structure as its base, made a transparent strain sensor detecting the motion of a finger. Various studies designed functionalized fabric to measure triboelectric charges induced by fabric deformation, making it possible to differentiate joint movement types (extension, flexion) and movement speed with the loose-fitting cloth. All these strain sensors require complicated chemical procedure and special techniques, which entail a higher manufacturing cost. Applicants' work differs in that Applicants examine the use of off-the-shelf, low-cost fabrics for fine-grained joint motion sensing.

Additionally, fiber has also been explored for joint motion sensing. Some have incorporated conductive fibers into the fabric with elastic cord and sensed the joint angle according to the resistance change of conductive fibers. Others made use of optical fiber, which measures the relative angle in a rotating joint based on the intensity modulation of a laser beam propagating in a single-mode optical fiber. Additionally, other researchers developed a sensing system including a retractable reel, a string and a potentiometer.

This system estimates the joint angle accurately under fast movement settings with the help of the retractable reel. Applicants' work differs in that Applicants study a different approach using off-the-shelf, low-cost fabrics, which potentially can be more flexible, and more comfortable to wear compared to fibers and reels.

Other motion-reconstruction systems involving fabric sensing combine fabric sensors with other types of sensors. Examples include E-Skin by Xenon, on-body sensing system by FIGUR8 and super soft stretch sensor by stretch sense. Besides sensing fabric, E-Skin system places 3-axis accelerometers and 3-axis gyro sensors on the plastic part on the chest. Stretch sensor attaches an extra circuit unit at the end of its smart garments.

Others have used the wearable inertial measurement units (IMUs) to estimate the “movement skill”. FIGUR8 claims to use 9-axis inertial measuring unit in its products. Applicants' work differs in that Applicants reconstruct joint motion solely based on fabric sensing without the aid from other types of sensors.

### Example 1.21. Conclusion

In this Example, Applicants studied the use of off-the-shelf conductive fabrics to sense the strain and pressure during joint motion and infer joint rotational angle. To achieve accurate and robust sensing, challenges arising from the properties of fabric materials are addressed. Applicants' work leveraged models from material science and biomechanics to characterize fabric properties and the relationship between skin deformation and joint angle. Applicants fabricated prototypes with off-the-shelf fabrics and a micro-controller. The system's sensing accuracy, robustness, and comfort are demonstrated by conducting system experiments and user studies with 10 participants.

Applicants currently fabricated prototypes only for the elbow joint. Moving forward, Applicants plan to fabricate prototypes for other types of body joints and examine the efficacy of the design. The general design principle still applies and will need to consider the anatomy of other joint types for possible adjustments on inferring joint angle. For joints such as wrists with larger degrees of freedom during rotation and capable of performing more types of rotational movement, Applicants consider optimizing the current design and arrangement of sensing fabrics to maximize the number of rotation types the system is capable of tracking.

Applicants envision thinner fabrics to help the body better dissipate heat. Also, Applicants consider arranging insulative layers close to skin to protect sensing fabrics and the few conductive threads connecting to the micro-controller from sweat.

The current fabrication of the fabric prototype involves lengthy human efforts. Applicants are interested in examining possible automation of certain steps to expedite the fabrication process. Most fabrication efforts have been on cutting, folding, and sewing the fabrics, which potentially can be automated by machines for mass production. The assembling of various fabric pieces needs only knowledge on the typical size of the bulge of the joint to ensure that sensing fabrics are properly placed to sense skin deformation and pressure. Since the fabric prototype will be fabricated in a small number of size options, for users fitting in the same size, their individual differences in the size of joint bulge would be small and can be handled by the dual sensing design (see above) to ensure sensing robustness. Thus, the fabric assembling does not need to be conducted on a per-user basis and can potentially be made repeatable. Applicants further envision that orthopedists at a local hospital can utilize the wearable systems of the present disclosure for monitoring joint recovery after surgeries.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of determining an angular motion in a subject, said method comprising:
applying a wearable system to a body region of the subject;
utilizing the wearable system to sense one or more parameters; and
correlating the one or more parameters to the angular motion in the subject.

2. The method of claim 1, wherein the one or more parameters are selected from the group consisting of resistance, pressure, deformation, skin deformation, strain, stress relaxation, and combinations thereof.

3. The method of claim 1, wherein the sensing comprises measuring strain of the wearable system.

4. The method of claim 3, wherein the measuring comprises sensing a change in resistance in the wearable system, wherein the change in resistance is caused by an alteration of a contact point in a portion of yarn of the wearable system, an alteration of contact pressure in a portion of yarn of the wearable system, or by an alteration of contact points and contact pressure of a portion of yarn of the wearable system.

5. The method of claim 1, wherein the sensing comprises measuring a change in pressure in the wearable system.

6. The method of claim 5, wherein the change in pressure is caused by at least one of yarn stretching, movement of the subject, movement at the body region, movement at a joint at the body region, and combinations thereof.

7. The method of claim 1, wherein the angular motion is selected from the group consisting of rotational motion, arbitrary motion, coarse motion, flexion, extension, motionless states, joint motion, joint rotation, muscle movement, rotational angle, joint rotational angle, and combinations thereof.

8. The method of claim 1, wherein the correlating comprises at least one of predicting angular motion, measuring angular motion, reconstructing angular motion, modeling angular motion, and combinations thereof.

9. The method of claim 1, wherein the correlating comprises utilizing a model selected from the group consisting of a model to compensate for stress relaxation during a motionless state of the subject, a model to characterize a geometric relationship between skin deformation and joint angle, a material science model, a biomechanical model, and combinations thereof.

10. The method of claim 1, wherein the correlating comprises:
monitoring resistance of the wearable system to infer muscle strain caused by joint motion;
recovering joint angle based, at least in part, on the monitoring; and
compensating for stress relaxation during a motionless state.

11. The method of claim 10, wherein the correlating further comprises:
characterizing a geometric relationship between skin deformation and joint angle; and
inferring joint rotational angle based, at least in part, on the characterization.

12. The method of claim 1, wherein the body region is selected from the group consisting of joints, muscles, a body extremity, a prehensile, an appendage of a body, an upper limb, a lower limb, a pivot joint, a hinge joint, a saddle joint, a ball-and-socket joint, a planar joint, an ellipsoidal joint, an elbow joint, a knee joint, an ankle joint, a wrist joint, a neck joint, a finger joint, a foot joint, a toe joint, a leg joint, an arm joint, and combinations thereof.

13. The method of claim 1, wherein the applying comprises wrapping the wearable system around a joint.

14. The method of claim 1, wherein the wearable system comprises a fabric, wherein the fabric is selected from the group consisting of a stretchable fabric, a conductive fabric, a stretchable and conductive fabric, a pressure sensitive fabric, a knitted fabric, and combinations thereof.

15. The method of claim 14, wherein the fabric lacks an electrode.

16. The method of claim 14, wherein the fabric comprises a plurality of fabrics, wherein the plurality of fabrics comprises a first fabric operable to sense a first parameter of the one or more parameters, and a second fabric operable to sense a second parameter of the one or more parameters.

17. The method of claim 16, wherein the first fabric is operable to sense deformation and the second fabric is operable to sense pressure.

18. The method of claim 17, wherein the first fabric reacts to different levels of strain with varying resistance to sense deformation and the second fabric augments the first fabric by sensing pressure from the body region during motion.

19. The method of claim 14, wherein the fabric comprises one or more layers, where the one or more layers comprise a first layer that faces a portion of skin and a second layer positioned above the first layer.

20. The method of claim 19, wherein the first layer senses pressure and the second layer senses deformation.

21. The method of claim 1, wherein the wearable system further comprises a microcontroller, wherein the microcontroller obtains data relating to the one or more parameters.

22. The method of claim 21, wherein the microcontroller resides outside of a fabric of the wearable system.
